(19) United States
(12) Patent Application Publication
(10) Pub. No.: US 2020/0165580 A1
(43) Pub. Date: May 28, 2020

(54) POLYPEPTIDES WITH POLYSACCHARIDE MONOOXYGENASE ACTIVITY AND USE THEREOF FOR THE PRODUCTION OF FERMENTABLE SUGARS

(71) Applicant: ABENGOA BIOENERGÍA NUEVAS TECNOLOGÍAS, S.A., Sevilla (ES)

(72) Inventors: Lucía MARTÍN PÉREZ, Sevilla (ES); Bruno DÍEZ GARCÍA, Sevilla (ES); Francisco Manuel REYES SOSA, Sevilla (ES); Noelia VALBUENA CRESPO, Sevilla (ES); Antonio Javier MORENO PÉREZ, Sevilla (ES); Dolores PÉREZ GÓMEZ, Sevilla (ES); Ana Isabel PLATERO GÓMEZ, Sevilla (ES); Sandra GAVALDÁ MARTÍN, Sevilla (ES); Laura VIÑAS DE LA CRUZ, Sevilla (ES); Javier ROCHA MARTÍN, Sevilla (ES); Laura SÁNCHEZ ZAMORANO, Sevilla (ES); María De Los Angeles BERMÚDEZ ALCÁNTARA, Sevilla (ES); Laura LEDESMA GARCÍA, Sevilla (ES); Juan Luis RAMOS MARTÍN, Sevilla (ES)

(21) Appl. No.: 16/481,815

(22) PCT Filed: Jan. 12, 2018
(86) PCT No.: PCT/ES2018/070023

§ 371 Date: Oct. 29, 2019

(30) Foreign Application Priority Data
Feb. 1, 2017 (ES) P 201730113

(51) Int. Cl. C12N 9/02; C12N 15/80; C12P 7/10; C12P 19/02

(52) U.S. Cl. CPC C12N 9/0071; C12P 19/02; C12P 7/10; C12N 15/80

(57) ABSTRACT

The invention refers to polypeptides with polysaccharide monooxygenase activity, to a host cell that expresses them, preferably a Myceliophthora thermophila cell that recombinantly expresses at least one of these polypeptides, to an enzymatic composition comprising at least one of these polypeptides, preferably together with other cellulolytic enzymes, the use of this host cell, of at least one of the polypeptides with polysaccharide monooxygenase activity or of the enzymatic composition for the degradation of cellulosic biomass and to a process for the production of bioproducts, preferably bioethanol, including the use of this host cell, of at least one of the polypeptides of the invention or of the enzymatic composition of the invention.

The invention relates to the field of bioproducts, more particularly to the biotechnological processes for biofuel production. Specifically, the invention relates to enzymes with polysaccharide monooxygenase activity and their use, as part of enzymatic cocktails, for the production of fermentable sugars by hydrolysis of cellulosic biomass during processes for the production of bioproducts such as bioethanol.

### PRIOR STATE OF THE ART

Biofuels are an attractive alternative to fossil fuels and can be obtained by fermenting monomeric sugars derived from starch or cellulose and hemicellulose.

Plant biomass provides an abundant source of potential energy in the form of carbohydrates that can be used for numerous industrial and agricultural processes and, therefore, is an important and renewable source for generating fermentable sugars. Fermentation of these sugars can produce valuable commercial end products, such as ethanol also known as bioethanol.

Although fermentation of sugars to ethanol is relatively straightforward, efficient conversion of cellulosic biomass to fermentable sugars, such as glucose, is more challenging. The huge potential energy of carbohydrates in plant biomass is not sufficiently used because the sugars form part of complex polymers (polysaccharides such as cellulose and hemicellulose) and, therefore, are not easily accessible for fermentation. Thus, cellulose can be pre-treated, mechanically, chemically, enzymatically or in other ways, to increase its susceptibility to hydrolysis. After this pre-treatment process, occurs a saccharification or hydrolysis stage consisting of an enzymatic process in which complex carbohydrates (such as starch or cellulose) are hydrolysed into their monosaccharide components. The goal of any saccharification technology therefore is to alter or eliminate structural and compositional impediments in order to improve the rate of enzymatic hydrolysis and increase the yields of fermentable sugars obtained from cellulose and hemicellulose (N. Mosier et al., 2005, Bioresource Technology 96, 673-686). After this stage of saccharification, a fermentation process takes place. Therefore, the higher the amount of complex sugars that remain at the end of the hydrolytic process, the lower the yield in ethanol production at the end of the fermentation process. Thus, an area of research directed at reducing costs and improving the yield of biofuel production processes is focused on improving the technical efficiency of hydrolytic enzymes, or generally on improving the efficiency of enzymatic cocktails used to generate, by hydrolysis, fermentable sugars from biomass.

It has been shown that individual enzymes are only able to partially digesting cellulose and hemicellulose and, therefore, the combined action of different classes of enzymes is required to complete their conversion into monomeric sugars. Many more enzymes are required for digesting hemicellulose to monomeric sugars than cellulose, including enzymes with xylanase, beta-xylosidase, arabinofuranosidase, mannanase, galactosidase, and glucuronidase activity. Other enzymes without glycosyl hydrolase activity, such as acetyl xylan esterase and ferulic acid esterase, may also be involved. Therefore, the enzymatic hydrolysis of polysaccharides for conversion into soluble sugars and, finally, into monomers such as xylose, glucose and other pentoses and hexoses, is catalysed by several enzymes which are collectively called “cellulases”. Cellulases comprise at least three main activities, endo-β-glucanase (EC 3.2.1.4), exo-β-glucanase or cellobiohydrolase (EC 3.2.1.9.1) and β-glucosidase (EC 3.2.1.21), and it has been shown that they act synergistically in cellulose hydrolysis (Woodward, J. 1991, Bioresource Technology Vol. 36, pg. 67-75).

Microbial cellulases, particularly those of fungal origin, have become important biocatalysts due to their extensive industrial applications (Kuhad R. C. et al., 2011, Enzyme Research, Article ID 280696). Nowadays, considerable attention has been paid to research on cellulases and the challenges in their production, especially for the improvement of the economy of the process for their industrial application, in order to obtain cells with greater activity and better properties.

On the other hand, the glycosyl-hydrolase proteins of the family 61 (GH61) have been known for more than 20 years. The first GH61 described, called CEL1, was isolated from Agaricus bisporus in 1992 (Raguz et al., 1992, Gene 119: 183-190). These GH61 proteins are accessory proteins that contribute to the cellulose degradation. The fact that these enzymes act by direct oxidation of cellulose, rather than by hydrolysis, has led to their current name: Cu dependent polysaccharide monooxygenases (polysaccharide monooxygenases; PMOs). Compared to other cellulolytic enzymes, PMOs are relatively small proteins with typical molecular masses between 20 and 50 kDa (Baldrian and Valaskova 2008, FEMS Microbiology Reviews 32: 501-521; Harris et al, 2010, Biochemistry 49: 3305-3316). These proteins require an oxygen molecule to break the substrate by oxidation. One of the two atoms ends up forming a water molecule, and with the other one the direct oxidation of the substrate is performed. Therefore, the members of this enzyme family act as Cu monooxygenases that catalyse cellulose breakdown by an oxidative mechanism, releasing cellodextrins (Langston et al., 2011, Applied and Environmental Microbiology 77: 7007-7015). The action of PMOs is also described, for example, in Glyn R. Hemsworth, et al., 2014, Nat Chem Biol. 10(2): 122-126, in Lucia Zifcakova, Petr Baldrian, 2012, Fungal ecology 5: 481-189 and in many patent documents where new PMOs have been identified in filamentous fungi, as in P201430155.

The hydrolytic efficiency of a multi-enzymatic complex in the saccharification process of cellulosic material depends on the properties of the individual enzymes present in the complex Therefore, in the context of biofuel production processes, it is necessary to design enzymatic cocktails with improved individual activities whose use during the saccharification or hydrolysis stage of cellulosic biomass leads to an improvement in the yield of this stage through an increase in the amount of fermentable sugars obtained. Then, these sugars can be fermented to produce biofuels, such as bioethanol, so the use of these improved enzymatic cocktails would ultimately increase the efficiency and profitability of the whole biofuel production process.

### DESCRIPTION OF THE INVENTION

The present invention relates polypeptides with polysaccharide monooxygenase activity (PMO, also known as glycosyl-hydrolase of the family 61 or GH61) which have been identified, isolated and characterised from different microorganisms. As the examples of the present invention show, these polypeptides have the advantage that they are able to increasing the saccharification yield of cellulosic biomass, by increasing the amount of fermentable sugars (mainly glucose) produced at the end of this hydrolytic process, when they are added to the enzymatic cocktails normally used in biofuel production processes.

These polypeptides are polysaccharide monooxygenase enzymes that intervene in the initial stages of the decomposition process from cellulosic biomass to fermentable sugars, these enzymes being responsible for improving the accessibility of the rest of the enzyme machinery to the substrate. In this way, they increase the yield of the hydrolysis process by increasing the amount of simple sugars obtained (mainly glucose) and thus, ultimately, the yield of ethanol production from biomass.

Specifically, the inventors of the present invention have identified, in the genome of different fungi, 22 genes that encode enzymes with PMO activity. The amino acid sequences of these 22 mature enzymes are shown in the SEQ ID NO: 49 to SEQ ID NO:SEQ ID NO: 70. These polypeptides have been isolated from Byssochlamys spectabilis, Penicillium oxalicum 114-2, Aspergillus clavatus, Aspergillus fumigatus Af293, Aspergillus niger CBS 513.88, Aspergillus niger ATCC 1015, Aspergillus ruber CBS135680, Aspergillus terreus NIH2624, Neosartorya fischeri NRRL181, Aspergillus kawachii IFO 4308, Aspergillus nidulans FGSC A4, Aspergillus oryzae RIB40, Baudoinia compniacensis UAMH 10762, Penicillium roqueforti FM164, Sclerotinia sclerotiorum, Penicillium italicum, Aspergillus flavus, Penicillium crysogenum Wisconsin, Penicillium expansum and Penicillium rubens Wisconsin. These polypeptides have been characterised and expressed recombinantly in a host cell, preferably in the C1 strain of Myceliophthora thermophila, which secretes them to the extracellular medium together with an enzymatic mixture comprising the main cellulolytic enzymes. Thus, the effect of these PMOs of the invention on the yield of these cellulolytic enzymatic cocktails in the saccharification of cellulosic biomass, preferably pre-treated and more preferably on PCS or pre-treated corn stover, has been evaluated. The results have shown an increase in the concentration of fermentable sugars (mainly glucose) released in the hydrolytic process compared to an enzymatic cocktail that did not comprise the polypeptides with PMO active of the invention.

Thus, this invention demonstrates the improvement in the enzymatic hydrolysis of cellulosic biomass with a cellulolytic cocktail produced by a host cell, preferably M. thermophila, modified to recombinantly express at least one of the polypeptides with PMO activity of the invention.

Improving the yield of enzymatic mixtures used to produce fermentable sugars from cellulosic material in biofuel production processes, preferably ethanol, is essential to ensure the profitability of these processes. For this reason, it is important to find new and improved enzymes capable of increasing the efficiency (production yield of fermentable sugars during hydrolysis) of the enzymatic cocktails in which they are included. The supplementation of an enzymatic cocktail comprising cellulolytic enzymes with these PMOs provided by the invention contributes, therefore, to improving the yield of the hydrolytic process where these cocktails are used, in particular by increasing the yield of glucose release from biomass.

Therefore, a first aspect of the present invention relates to an isolated polypeptide, preferably with polysaccharide monooxygenase activity, hereinafter referred to as the “polypeptide of the invention” or the “PMO of the invention”, comprising an amino acid sequence having at least 80% identity with an amino acid sequence selected from SEQ ID NO:SEQ ID NO: 49 to SEQ ID NO:SEQ ID NO: 70, preferably selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably with SEQ ID NO: 65.

The polypeptide of the invention may be isolated from its natural source (microorganism producer) or environment (extracellular medium), preferably after its natural expression in the fungi indicated later in this description or from a natural or growing environment where such fungi are present, or it may be recombinantly produced. The polypeptide of the present invention and its variants or derivatives can be synthesized, for example, but not be limited to, in vitro. For example, by solid-phase peptide synthesis or recombinant DNA approximations. The polypeptide of the invention can be produced recombinantly, not only directly but also as a fusion polypeptide linked to another peptide or polypeptide which can be, but not be limited to, heterologous (with different origin than the polypeptide of the invention). This other peptide or polypeptide fused to the polypeptide of the invention may comprise or consist of, for example but not be limited to, a signal sequence (signal peptide) or another polypeptide which has for example a sequence to facilitate its expression and/or purification or a protease cleavage site, for example but not be limited to, at the N-terminal end of the mature protein.

The polypeptide of the invention may have variants. These variants refer to limited variations in the amino acid sequence, which allow the maintenance of the functionality of the polypeptide. This means that the reference sequence and the variant sequence are similar as a whole, and identical in many regions. These variations are generated by substitutions, deletions or additions. Such substitutions are preferably by conserved amino acids. Conserved amino acids are amino acids that have side chains and similar properties in terms of, for example, hydrophobicity or aromaticity. These substitutions include, but are not limited to, substitutions between glutamic acid (Glu) and aspartic acid (Asp), between lysine (Lys) and arginine (Arg), between asparagine (Asn) and glutamine (Gln), between serine (Ser) and threonine (Thr), and/or between the amino acids that compose the group alanine (Ala), leucine (Leu), valine (Val) and isoleucine (lie). Variations can be artificially generated variations such as mutagenesis or direct synthesis. These variations do not lead to essential changes in the essential characteristics or properties of the polypeptide. Therefore, within the extent of this invention are also included peptides or polypeptides whose sequence of amino acids is identical or homologous to the sequences described in this invention.

The terms “polysaccharide monooxygenase”, “PMO”, “Glycosylhydrolase 61 family” or “GH61” refer to an enzyme with GH61 or PMO activity, which when included in a saccharification reaction (for example, one in which endoglucanase, beta-glucosidases and celobiohydrolases are used), results in a higher amount (higher yield) of one or more soluble sugars (e.g. glucose) compared to the saccharification reaction carried out under the same conditions but in the absence of the GH61 protein. PMO activity can be determined by, for example, indirect oxidative assays that colorimetrically evidence the electron transfer phenomenon using different electron donor and acceptor compounds (Kitt et al., 2012, Biotechnology for Biofuels Vol. 5:79, pg. 1-13). On the other hand, PMO activity on biomass could be measured, for example, by combining the polypeptide to be tested with cellulose or cellulolytic enzymes in a saccharification reaction and determining whether there is an increase in glucose yield compared to the same saccharification reaction performed in the presence of the same enzymes and the same conditions but in the absence of the polypeptide to be tested.

In this invention, the PMOs comprising the SEQ ID NO: 49 to SEQ ID NO: 70 have been obtained from the following fungi:
SEQ ID NO: 49, Byssochlamys spectabilis.
SEQ ID NO: 50, Penicillium oxalicum 114-2.
SEQ ID NO: 51, Aspergillus clavatus.
SEQ ID NO: 52, Aspergillus fumigatus Af293.
SEQ ID NO: 53, Aspergillus niger CBS 513.88.
SEQ ID NO: 54, Aspergillus niger ATCC 1015.
SEQ ID NO: 55, Aspergillus ruber CBS135680.
SEQ ID NO: 56, Aspergillus terreus NIH2624.
SEQ ID NO: 57, Neosartorya fischeri NRRL181.
SEQ ID NO: 58, Aspergillus kawachii IFO 4308.
SEQ ID NO: 59, Aspergillus nidulans FGSC A4.
SEQ ID NO: 60, Aspergillus oryzae RIB40.
SEQ ID NO: 61, Aspergillus oryzae RIB40.
SEQ ID NO: 62, Baudoinia compniacensis UAMH 10762.
SEQ ID NO: 63, Penicillium roqueforti FM164.
SEQ ID NO: 64, Sclerotinia sclerotiorum.
SEQ ID NO: 65, Penicillium roqueforti FM164.
SEQ ID NO: 66, Penicillium italicum.
SEQ ID NO: 67, Aspergillus flavus.
SEQ ID NO: 68, Penicillium crysogenum Wisconsin.
SEQ ID NO: 69, Penicillium expansum.
SEQ ID NO: 70, Penicillium rubens Wisconsin.

In a more preferred embodiment, the polypeptide of the invention comprises an amino acid sequence having at least 81%, 82%, 83%, 84%, 85%, 86%, 87%, 88%, 89%, 90%, 91%, 92%, 93%, 94%, 95%, 96%, 97%, 98% or99% identity with an amino acid sequence selected from SEQ ID NO: 49 to SEQ ID NO: 70, preferably selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably with SEQ ID NO: 65. In an even more preferred embodiment, the polypeptide of the invention comprises an amino acid sequence that has at least 90% identity with an amino acid sequence selected from SEQ ID NO: 49 to SEQ ID NO: 70, preferably selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably with SEQ ID NO: 65.

The term “identity”, “homology” or “percentage of identity” refersto the ratio of residues of nucleic acids or amino acids that are identical between two sequences of nucleic acids or amino acids being compared. Preferably, “identity” refers to the ratio of residues of nucleic acids or amino acids that are identical between two nucleic acid or amino acid sequences, with respect to a length of at least 100 residues, preferably with respect to the full length of the reference sequence. The degree of identity can be determined by the Clustal method, the Wilbur-Lipman method, the GAG program, which includes GAP, BLAST or BLASTN, EMBOSS Needle and FASTA. In addition, the Smith Waterman algorithm can be used to determine the degree of identity between two sequences.

For sequence comparison, typically one of the sequences acts as a reference sequence with which the “problem” sequences are compared. When a sequence comparison algorithm is used to determine its identity, the reference sequence and the problem sequence(s) are entered into the program, and the program parameters are configured. The program parameters that appear by default can be used or configured; preferably these parameters will be those that appear by default. Thus, the sequence comparison algorithm calculates the percentage of identity between the problem sequence(s) and the reference sequence based on program parameters. Two examples of algorithms that are useful for determining percentage of sequence identity are BLAST and BLAST 2.0, described in Altschul et al. (1997) Nucleic Acids Res 25(17):3389-3402 and Altschul et al. (1990) J. Mol Biol 215(3)-403-410, respectively. Preferably, the degree of identity to which this invention refers is calculated by BLAST. The software for conducting the BLAST analysis is publicly available at the National Center for Biotechnology Information (NCBI).

In a more preferred embodiment, the polypeptide of the invention comprises a sequence of amino acids selected from SEQ ID NO: 49 to SEQ ID NO: 70, preferably selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably comprising the SEQ ID NO: 65.

The polypeptides of the invention include those that may comprise a signal peptide linked to its N-terminal end. This signal peptide can be the one that is naturally present in a PMO or another from another protein (heterologous). Examples of heterologous signal peptides that could be linked to the PMO polypeptides of the invention are, but not be limited to, the signal peptide of Aspergillus niger glucoamylase, preferably of sequence SEQ ID NO: 46, and the PMO-06230 peptide signal from M. thermophila, preferably from sequence SEQ ID NO: 48. More preferably, the signal peptide with SEQ ID NO: 46 is encoded by the nucleotide sequence SEQ ID NO: 45 and the signal peptide with SEQ ID NO: 48 is encoded by the nucleotide sequence SEQ ID NO: 47.

When the polypeptide of the invention is linked to a signal peptide, said polypeptide corresponds to the pre-protein or immature polypeptide of the mature PMO enzyme. Thus, both mature PMO polypeptides (where there is no signal peptide) and these immature PMO pre-proteins or polypeptides that comprising a signal peptide located at the N-terminal end of the amino acid sequence of the mature enzyme, are within the extent of this invention. These pre-proteins are preferably the SEQ ID NO: 23 to SEQ ID NO: 44. The signal peptide of each of these sequences SEQ ID NO: 23 to SEQ ID NO: 44 is shown in the respective polypeptide sequences in the sequence listing.

In an even more preferred embodiment, the polypeptide of the invention consists of an amino acid sequence selected from SEQ ID NO: 49 to SEQ ID NO: 70, preferably selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably it consists of the SEQ ID NO: 65. These sequences correspond to the mature polypeptides of the invention.

In another preferred embodiment, the polypeptide of the invention consists of an amino acid sequence selected from SEQ ID NO: 23 to SEQ ID NO: 44, preferably selected from SEQ ID NO: 26, SEQ ID NO: 27, SEQ ID NO: 33, SEQ ID NO: 34 or SEQ ID NO: 39; more preferably selected from SEQ ID NO: 26, SEQ ID NO: 33 or SEQ ID NO: 39; even more preferably it consists of the SEQ ID NO: 39. These sequences correspond to the immature polypeptides of the invention (comprising the native or naturally present signal peptide in the enzyme).

In another preferred embodiment, the polypeptide of the invention further comprises, linked to its N-terminal end, a signal peptide. Preferably such a signal peptide is selected from the list consisting of: the native signal peptide or that originally present in the enzyme naturally (SPWT, whose amino acid sequence is indicated in the listing of sequences in each polypeptide of the invention of SEQ ID NO: 23 to SEQ ID NO: 44), the glucoamylase A signal peptide of A. niger (SPGA, preferably of SEQ ID NO: 46) or the PMO-06230 peptide signal from M. thermophila (SPPMO, preferably from SEQ ID NO: 48). In a more preferred embodiment, the polypeptide of the invention is linked, to its N-terminal end, to the SPGA signal peptide, more preferably with SEQ ID NO: 46.

As explained above, the term “pre-protein” refers to a polypeptide that includes a signal peptide (or leading sequence) at its terminal amino end. This signal peptide is cleaved from the pre-protein by a peptidase, thus secreting the mature protein. The secreted portion of the polypeptide is called “mature protein” or “secreted protein”. The “signal peptide” is the one that directs the polypeptide inside the cell towards its secretion pathway.

As the examples of this invention show, the polypeptides referred to here as SEQ ID NO: 52, SEQ ID NO: 53, SEQ ID NO: 59, SEQ ID NO: 60 and SEQ ID NO: 65 were those which were provided the largest increases in saccharification capacity when were added as a supplement to enzymatic mixtures or cocktails produced by M. thermophila (see FIGS. 1 and 2). Therefore, in a more preferred embodiment, the polypeptide of the invention consists of an amino acid sequence selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably it consists of the SEQ ID NO: 65.

According to the examples shown below, the polypeptides of the invention provided increases in saccharification capacity (measured as relative units of released glucose) following the order set out below from highest to lowest:
SEQ ID NO: 65
SEQ ID NO: 59
SEQ ID NO: 52
SEQ ID NO: 60
SEQ ID NO: 53
SEQ ID NO: 55
SEQ ID NO: 51
SEQ ID NO: 58
SEQ ID NO: 64
SEQ ID NO: 54
SEQ ID NO: 56
SEQ ID NO: 66
SEQ ID NO: 68
SEQ ID NO: 69
SEQ ID NO: 70
SEQ ID NO: 49
SEQ ID NO: 61
SEQ ID NO: 62
SEQ ID NO: 50
SEQ ID NO: 57
SEQ ID NO: 63
SEQ ID NO: 67

Therefore, the above list shows the order of preference of the polypeptides of the invention.

In a particular embodiment, the polypeptide of the invention comprises an amino acid sequence having at least 80% identity, preferably at least 90% identity, with the SEQ ID NO: 65 and is linked, to its N-terminal end, to the SPGA signal peptide, more preferably to SEQ ID NO: 46.

The term “increase” as used in the present invention refers to an increase in the yield of a reaction product, for example, from a fermentable sugar, preferably glucose, produced when a particular component present during the reaction (such as a PMO polypeptide of the invention) causes a greater production of the product compared to a reaction performed under the same conditions and with the same substrate but in the absence of the component in question.

Due to the degeneration of the genetic code, in which several nucleotide triplets give rise to the same amino acid, there are several nucleotide sequences that give rise to the same amino acid sequence. For this reason, another aspect of the invention refers to an isolated polynucleotide, hereinafter “polynucleotide of the invention”, which encodes any of the polypeptides of the invention. Another aspect of the invention refers to an isolated polynucleotide comprising a nucleotide sequence complementary to the polynucleotide of the invention, or hybridizing under a stringent conditions, preferably under conditions of high astringency, with the polynucleotide of the invention.

The terms “nucleotide sequence”, “nucleic acid”, “oligonucleotide” and “polynucleotide” are used interchangeably herein and refer to a polymeric form of nucleotides of any length that may or may not be, chemically or biochemically modified. Therefore, they refer to any polyribonucleotide or polydesoxyribonucleotide, whether single-chain or double-stranded. The polynucleotide of the invention, therefore, can be DNA, RNA, or derivatives of both DNA and RNA, including cDNA. The polynucleotide of the invention can be obtained artificially by conventional cloning and selection methods, or by sequencing. The polynucleotide, in addition to the coding sequence, may comprise other elements, for example but not limited to, one or more introns, non-coding sequences at the 5′ and/or 3′ ends, ribosome binding sites, coding sequences for a signal peptide, or stabilising sequences. These polynucleotides may additionally include coding sequences for additional amino acids that may be useful, for example, but not limited, to increase the stability of the peptide generated from it or to allow better purification of the peptide.

The nucleic acid sequences encoding the polypeptides of the invention, for example, can be designed on the basis of the amino acid sequences provided in the present invention. The nucleotide coding sequences for the immature PMO polypeptides described in the present invention (SEQ ID NO: 23 to SEQ ID NO: 44) consist, preferably, of SEQ ID NO: 1 to SEQ ID NO: 22. The nucleotide sequence coding for the signal peptide in each of these immature PMOs is shown in the respective sequences SEQ ID NO: 1 to SEQ ID NO: 22 of the sequence listing.

The polynucleotide of the invention can be introduced into a gene construct, for example, into a cloning vector or expression vector, to allow its replication or expression. Preferably, said vector is an appropriate vector for the expression and purification of the polypeptide of the invention. For this reason, another aspect of the invention refers to a gene construct that comprises:
the polynucleotide of the invention that encodes any of the polypeptides of the invention, or
a combination of those polynucleotides of the invention.

Hereinafter, this gene construct will be referred to as “gene construct of the invention” or “construct of the invention”.

The term “gene construct”, as used herein, refers to a nucleic acid molecule, both monocatenary and bicatenary, that is isolated and modified to contain nucleic acid segments in a way that could not exist in nature. The term “nucleic acid construct” or “gene construct” is synonymous with the term “expression cassette” when the nucleic acid construct contains the control sequences required for the expression of the polynucleotide of the invention. Therefore, the genetic construct of the invention may also comprise one or more control or regulatory sequences of gene expression, such as, but not limited to, promoter sequences, leader sequences, terminator sequences of transcription, polyadenylation sequences, signal sequences, regulators, enhancers, etc.

The term “control sequences” includes all components that are necessary or advantageous for the expression of the polynucleotide of this invention in a cell. Each control sequence may be of the same or different origin as the polynucleotide of the invention. Such control sequences include, but are not limited to, a leader sequence, a booster or regulator sequence, a polyadenylation sequence, a pro-peptide sequence, a promoter, a coding sequence for a signal peptide, and a transcription terminator sequence. At a minimum, control sequences include a sequence of the signal peptide, and more preferably also a promoter, and termination signals from the transcription and translation. Control sequences with linkers may also be provided in order to introduce specific restriction sites that facilitate the binding of the control sequences to the coding region of the polypeptide of the invention. Appropriate control sequences for the expression of a polynucleotide in eukaryotic cells are known in the state of the art.

As used herein, the term “promoter” refers to a nucleotide sequence, usually “upstream” of the transcription starting point, that is capable of initiating transcription in a cell. This term includes, but is not limited to, constitutive promoters, specific cellular promoters, ubiquitous promoters, and inducible or repressible promoters. In general, control sequences depend on the origin of the host cell into which the gene construct is to be inserted.

In a preferred embodiment, the gene construct of the invention is an expression vector. An “expression vector” is a linear or circular DNA molecule comprising at least one polynucleotide of the invention operationally linked to additional nucleotides provided for its expression. This vector that comprising the polynucleotide of the invention ca n be introduced into a host cell in such a way that the vector is maintained as a chromosomal component or as an autoreplicating extracromosomal vector.

The term “operatively linked” refers to a configuration in which a control sequence is located in an appropriate position with respect to the coding sequence of the polynucleotide of the invention, such that the control sequence directs the expression of said polynucleotide. When creating the expression vector, the coding sequence is located in the vector in such a way that it is operatively linked to the appropriate control sequences for its expression. Therefore, the expression vectors referred to in the present invention comprise the polynucleotide of the invention, a promoter, and transcription and translation termination signals. The various nucleic acids and control sequences described herein may link together to produce a recombinant expression vector which may also include one or more suitable restriction sites to allow insertion or substitution of the polynucleotide encoding for the polypeptide of the invention at such sites.

The expression vector referred to in the present invention may be any vector (e.g. plasmid or virus) that can be conveniently subjected to a recombinant DNA procedure and can produce the expression of the polynucleotide of the invention. The choice of vector will normally depend on the compatibility of the vector with the host cell into which the vector is to be introduced. The expression vector can be, for example but not be limited to, a plasmid, a cosmid, a phage, a virus or viral vector, an artificial bacterial chromosome (BAC), an artificial yeast chromosome (YAC), or similar.

Vectors can be linear or closed circular. The vector can be an autonomous replicating vector, in other words, a vector that exists as an extracromosomal entity, whose replication is independent of chromosomal replication, for example, a plasmid, an extracromosomal element, a mini chromosome, or an artificial chromosome. The vector can contain any means to ensure self-replication. Alternatively, the vector may be of the type that, when introduced into the host cell, it integrates into the genome and replicates along with the chromosome(s) in which it has been integrated. In addition, a single vector or plasmid or two or more vectors or plasmids together containing the total DNA to be introduced into the host cell genome, or a transposon, may be used.

The vectors used in this invention contain, preferably, one or more selectable markers that allow an easy selection of transformed, transfected, transduced, or similar cells. A selectable marker is a gene whose product provides a distinguishable signal or effect, such as but not limited to, luminescence, resistance to biocides or virus, resistance to heavy metals, prototrophy to auxotrophs and similar. Markers selectable for use in a host filamentous fungus cell include, but are not limited to, amdS (acetamidase), argB (ornithine carbamoyltransferase), bar (phosphinothricin acetyltransferase), hph (higromicin phosphotransferase), niaD (nitrate reductase), pyrG (orotidine-5′-phosphate decarboxylase), pyr5, pyr4, cysC (sulphate adenyltransferase), and trpC (anthranylate synthase), as well as their equivalents.

The vectors referred to in the present invention preferably contain an element(s) allowing the integration of the vector into the genome of the host cell or the autonomous replication of the vector into the cell independently of the genome. For integration into the host cell genome, the vector may be based on the polynucleotide sequence of the invention or on any other vector element for integration into the genome by homologous or non-homologous recombination. Alternatively, the vector may contain additional nucleotide sequences to direct integration by homologous recombination into the host cell genome at a precise location(s) of the chromosome(s).

For autonomous replication, the vector may comprise a replication origin that allows it to replicate autonomously in the host cell in question. The replication origin can be any plasmid replicator that mediates autonomous replication that works in a cell. The term “replication origin” or “plasmid replicator” is defined herein as a nucleotide sequence that allows a plasmid or vector to replicate in vivo. Examples of useful replication origins in a filamentous fungus cell are AMA1 and ANS1.

A single polynucleotide of the invention or several polynucleotides of the invention may be inserted jointly into the host cell by introducing one or more expression vectors. Similarly, both one and more than one copy of the same polynucleotide of this invention can be inserted into the host cell to increase the production of the gene product(s). An increase in the number of copies of the polynucleotide can be obtained by integrating at least one additional copy of the sequence into the host cell genome and by including with the polynucleotide an amplifiable selectable marker gene, where cells containing amplified copies of the selectable marker gene, and thus additional copies of the polynucleotide, can be selected by culturing the cells in the presence of the appropriate selection agent.

The procedures used to link the elements described above to construct the recombinant expression vectors referred to in this invention are well known to technical experts.

The gene construct of the invention may be introduced into a host cell competent to carry out the expression of one or more of the polypeptides of the invention. Thus, another aspect of the invention refers a host cell which comprises, in a recombinant manner (i.e. unnatural or by human intervention), one or more of the polynucleotides of the invention or the gene construct of the invention, hereinafter referred to as the “host cell of the invention”. In other words, the host cell of the invention has been transformed, transfected, transduced or similar, with at least one polynucleotide of the invention or with the gene construct of the invention. Preferably, the host cell of the invention is not a Penicillium roqueforti cell, and more preferably not a Byssochlamys spectabilis, Penicillium oxalicum, Aspergillus clavatus, Aspergillus fumigatus, Aspergillus niger, Aspergillus ruber, Aspergillus terreus, Neosartorya fischeri, Aspergillus kawachii, Aspergillus nidulans, Aspergillus oryzae, Baudoinia compniacensis, Penicillium roqueforti, Sclerotinia sclerotiorum, Penicillium italicum, Aspergillus flavus, Penicillium crysogenum, Penicillium expansum or Penicillium rubens cell. Preferably, the host cell of the invention comprises, in a recombinant manner (i.e. introduced by human intervention), one or more of the polynucleotides of the invention selected from the list consisting of the SEQ ID NO: 1 to SEQ ID NO: 22, preferably one or more of the polynucleotides of SEQ ID NO: 4, SEQ ID NO: 5, SEQ ID NO: 11, SEQ ID NO: 12 or SEQ ID NO: 17; more preferably from SEQ ID NO: 4, SEQ ID NO: 11 or SEQ ID NO: 17; even more preferably from SEQ ID NO: 17.

In another preferred embodiment, the polynucleotide of the invention encodes for a polypeptide of the invention comprising a sequence of amino acids presenting at least 80% identity, more preferably at least 90% identity, with the SEQ ID NO: 52, SEQ ID NO: 53, SEQ ID NO: 59, SEQ ID NO: 60 or SEQ ID NO: 65; more preferably with SEQ ID NO: 52, SEQ ID NO: 59 or SEQ ID NO: 65; even more preferably with SEQ ID NO: 65.

In another preferred embodiment, the gene construct of the invention comprises at least one polynucleotide of the invention coding for a polypeptide of the invention comprising a sequence of amino acids presenting at least 80% identity, more preferably at least 90% identity, with the SEQ ID NO: 52, SEQ ID NO: 53, SEQ ID NO: 59, SEQ ID NO: 60 or SEQ ID NO: 65; more preferably with SEQ ID NO: 52, SEQ ID NO: 59 or SEQ ID NO: 65; even more preferably with SEQ ID NO: 65.

In another preferred embodiment, the cell of the invention comprises at least one polynucleotide of the invention, or a gene construct of the invention comprising it, coding for a polypeptide of the invention comprising a sequence of amino acids presenting at least 80% identity, more preferably at least 90% identity, with the SEQ ID NO: 52, SEQ ID NO: 53, SEQ ID NO: 59, SEQ ID NO: 60 or SEQ ID NO: 65; more preferably with SEQ ID NO: 52, SEQ ID NO: 59 or SEQ ID NO: 65; even more preferably with SEQ ID NO: 65.

The “host cell”, as used here, includes any type of cell that is susceptible to transformation, transfection, transduction, and the like, with the polynucleotide of the invention or with the gene construct of the invention. The host cell may be eukaryotic, such as a mammalian cell, insect, plant, or fungus. In a preferred embodiment, the host cell is a filamentous fungus cell. Filamentous fungi are generally characterised by a mycelial wall composed of chitin, cellulose, glucan, chitosan, mannan, and other complex polysaccharides. In a more preferred embodiment, the host cell of the filamentous fungus is a Acremonium, Aspergillus, Aureobasidium, Bjerkandera, Ceriporiopsis, Coprinus, Coriolus, Cryptococcus, Filibasidium, Fusarium, Gibberella, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a more preferred embodiment, the host cell of the filamentous fungus is an Aspergillus awamori, Aspergillus foetidus or Aspergillus japonicus cell. In another more preferred embodiment, the host cell of the filamentous fungus is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium pseudograminearum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another more preferred embodiment, the host cell of the filamentous fungus is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Gibberella zeae, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell. In an even more preferred embodiment, the host cell of the invention is any strain of the species Myceliophthora thermophila. In an even more preferred embodiment, the host cell of the invention is the C1 strain of Myceliophthora thermophila.

It shall be understood that for the above species, the invention covers the perfect and imperfect states, and other taxonomic equivalents, e.g. anamorphs, with respect to the name of the species through which they are known. Technical experts will easily recognise the identity of suitable equivalents. For example, Myceliophthora thermophila is equivalent to Chrysosporium lucknowense.

The host cell of the invention comprises, therefore, at least one polynucleotide of the recombinantly introduced invention, preferably by means of the gene construct of the invention. The term “recombinantly introduced” refers to the fact that the polynucleotide of the invention or the gene construct of the invention is not naturally present in that cell, but has been intentionally introduced through genetic engineering procedures. These polynucleotides can encode the mature polypeptide or a pre-protein consisting of a signal peptide linked to the mature enzyme that will have to be processed later in order to produce the mature PMO enzyme.

The host cell of the invention expresses, and preferably also secretes to the extracellular medium, at least one of the PMO polypeptides of the invention, or any combination thereof, so that they are functional. The term “functional” refers that the expressed enzyme(s) retains its ability to oxidise cellulose, i.e., retains its polysaccharide monooxygenase activity. This activity can be measured by any appropriate procedure known in the state of the art to evaluate the activity of PMO.

The term “expression” includes any stage involved in the production of the polypeptide of the invention for example, but not limited to, transcription, post-transcriptional modification, translation, post-translational modification, and secretion.

The expression of the polypeptide of the invention in the host cell of the invention may be induced by any procedure known in the technique, such as the transformation of a suitable host cell with at least one polynucleotide of the invention or with the gene construct of the invention, and the culture of the transformed host cell underconditions that induce the expression of that polynucleotide in order to obtain the secreted and functional enzyme.

The host cell of the invention can be cultivated in a suitable nutrient medium, whether solid or liquid, for the production of the PMO polypeptides of the invention, using well known procedures in the technique. For example, the cell can be cultured by flask culture with agitation, and small-scale or large-scale fermentation (including continuous, batch or discontinuous, fed-batch, or solid-state fermentation) carried out in a laboratory or industrial bioreactor in a suitable medium and under conditions that allow PMO to be expressed and/or isolated. Cultivation takes place in a suitable nutrient medium comprising, for example, sources of carbon and nitrogen and inorganic salts, using the procedures known in the technique. Once the PMO has been secreted into the nutrient medium, it can be recovered directly from the medium.

Expressed PMO can be detected using procedures known in the technique specific to polypeptides. These detection procedures may include the use of specific antibodies, monitoring the formation of an enzyme product, or monitoring the disappearance of an enzyme substrate.

The resulting PMO can be retrieved from the medium using procedures known in the technique. For example, PMO can be recovered from the nutrient medium by conventional procedures including, but not limited to, centrifugation, filtration, extraction, spray drying, evaporation, or precipitation.

The PMOs produced in this invention can be purified by a variety of procedures known in the technique including, but not limited to, chromatography (e.g., ion exchange, affinity, hydrophobia, chromatofocus, and size exclusion), electrophoretic procedures (e.g. preparative isoelectric focusing), differential solubility (e.g. ammonium sulphate precipitation), SDS-PAGE, or extraction, in order to obtain a substantially pure PMO that can be included in an enzymatic composition together with other cellulolytic enzymes.

The host cell of the invention expresses at least one of the polypeptides of the invention, preferably one or more polypeptides comprising or consisting of the SEQ ID NO: 52, SEQ ID NO: 53, SEQ ID NO: 59, SEQ ID NO: 60 or SEQ ID NO: 65; more preferably one or more polypeptides comprising or consisting of the SEQ ID NO: 52, SEQ ID NO: 59 or SEQ ID NO: 65; even more preferably a polypeptide comprising or consisting of the SEQ ID NO: 65, or any combination of them, or all of them. The cell may also express one or more other cellulolytic, native or recombinant enzymes.

Another aspect of the invention concerns an enzymatic composition or enzymatic cocktail comprising at least one of the polypeptides of the invention, hereinafter referred to as the “composition of the invention”. Preferably, the composition of the invention comprises one or more polypeptides which comprise or consist of a polypeptide selected from the list consisting of the SEQ ID NO: 49 to SEQ ID NO: 70, preferably selected from SEQ ID NO: 52, SEQ ID NO: 53, SEQ ID NO: 59, SEQ ID NO: 60 or SEQ ID NO: 65; more preferably selected from SEQ ID NO: 52, SEQ ID NO: 59 or SEQ ID NO: 65; even more preferably the polypeptide from SEQ ID NO: 65.

“Enzymatic composition” or “enzymatic cocktail” means a mixture comprising at least two enzymes capable of hydrolysing, oxidising, degrading or similar, sugars.

This composition of the invention may further comprises other enzymatic activities, such as aminopeptidase, amylase, carbohydrase, carboxypeptidase, catalase, cellulase, such as endoglucanase, beta-glucosidase and/or celobiohydrolase; chitinase, cutinase, cyclodextrin glucosyltransferase, deoxyribonuclease, esterase, alpha-galactosidase, beta-galactosidase, glucoamylase, alpha-glucosidase, haloperoxidase, invertase, lacase, lipase, manosidase, oxidase, reductase, pectinolytic enzyme, peptidoglutaminase, peroxidase, phytase, polyphenoloxidase, protease, ribonuclease, transglutaminase, or xylanase, or any combination thereof. The additional enzyme(s) may be produced recombinantly or naturally, for example, by a microorganism belonging to the genus Aspergillus, such as Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, such as Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium pseudograminearum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Gibberella, such as Gibberella zeae; Humicola, such as Humicola insolens or Humicola lanuginosa; Trichoderma, such as Trichoderma harzianum, Trichoderma koningii, Trichoderma longibrachiatum, Trichoderma reesei, or Trichoderma viride; or Myceliophthora, such as Myceliophthora thermophila.

In a preferred embodiment, the composition of the invention also comprises another (one or more) cellulolytic enzyme. The term “cellulolytic enzymes” also known as “cellulases” refers to a class of enzymes capable of hydrolysing cellulose (links from P3-1,4-glucan or β-D-glucosidics) or hemicellulose to shorter oligosaccharides, cellobiose and/or glucose. Examples of cellulolytic enzymes include, but are not limited to, endoglucanases, beta-glucosidases, celobiohydrolases, polysaccharide monooxygenases (other than those described in the present invention), beta-xylosidases, endoxyloglucanases or endoxylanases. Thus, in a more preferred embodiment, these cellulolytic enzymes are selected from the list consisting of endoglucanases, beta-glucosidases, celobiohydrolases, celobiose dehydrogenases, polysaccharide monooxygenases, beta-xylosidases, alpha-xylosidases, endoxylanases, endoxyl glucanases, or any of their combinations. These cellulolytic enzymes may derive from the host cell of the invention or from other microorganisms producing cellulolytic enzymes other than the host cell of the invention, such as those indicated above. They can also be produced naturally or recombinantly.

In a preferred embodiment, the composition of the invention comprises all the PMO polypeptides of the invention, more preferably mature (without signal peptides), even more preferably of sequences SEQ ID NO: 49 to SEQ ID NO: 70.

Preferably, the composition of the invention comprises at least one polypeptide of the invention and other cellulolyticenzymes derived from the host cell of the invention. In an even more preferred embodiment, the composition of the invention is an enzymatic mixture expressed (secreted or obtained) by the host cell of the invention. In an even more preferred embodiment, the composition of the invention is an enzymatic mixture obtained by culture of the host cell of the invention, more preferably of the strain M. thermophila C1. This enzyme mixture expressed by the host cell of the invention shall comprise all or part of the cellulolytic enzymes produced naturally or recombinantly and secreted by that cell. These cellulolytic enzymes include, for example but not limited to, endoglucanases, beta-glucosidases, arabinofuranosidases, celobiohydrolases, celobiose dehydrogenases, xylidases, xylanases, etc.

The term “endoglucanase” or “EG” refers to a group of cellulase enzymes classified as E.C. 3.2.1.4. These enzymes hydrolyse the 3-1,4 internal glycosidic links of cellulose.

The term “cellobiohydrolase” refers to a protein that catalyses the hydrolysis of cellulose to cellobiose by exoglucanase activity, sequentially releasing cellobiose molecules from the reducing or non-reducing ends of cellulose or celooligosaccharides.

The term “beta-glucosidase”, as used herein, refers to an enzyme that catalyses the hydrolysis of a sugar dimer, including, but not limited to cellobiose, with the release of a corresponding sugar monomer, used, but not limited, for ethanol synthesis. The enzyme beta-glucosidase acts on the bridges β1->4 that bind two glucose molecules or substituted glucose (i.e. The disaccharide, cellobiose). It is an exocellulase with specificity for a variety of beta-D-glucoside substrates. It catalyses the hydrolysis of non-terminal reducing residues in beta-D-glucosides with glucose release.

The term “endoxylanase” refers to an enzyme that catalyses the endohydrolysis of 1.4-beta-D-xylosidic bonds in xylans.

The term “beta-xylosidase” refers to a protein that hydrolyses short 1.4-β-D-xyloligomers to xylose.

The term “endoxyloglucanase” refers to an enzyme specific to xyloglucan, capable of catalysing the solubilisation of xyloglucan in oligosaccharides but not showing substantial cellulolytic activity.

The term “cellobiose dehydrogenase” refers to an enzyme that catalyses a chemical reaction in which the substrates are cellobiose and the acceptor and the products are celobion-1.5-lactone and the reduced acceptor.

The term “alpha-xylosidase” refers to an enzyme that catalyses the hydrolysis of alpha-xylose residues that are not terminal reducers in alpha-xylosides.

In another preferred embodiment, the composition of the invention comprises at lea st one of the polypeptides of the invention and a cellulolytic mixture consisting of: endoglucanase, beta-glucosidase, celobiohydrolase I and celobiohydrolase II. More preferably, these cellulolytic enzymes come from M. thermophila.

In a more preferred embodiment, the composition of the invention also includes Cu.

The composition of the invention can be prepared according to known procedures in the technique and can be in liquid form or in a solid or semi-solid dry composition. For example, the composition may be in the form of a granulate or a microgranulate. The enzymes to be included in the composition may be stabilised according to the procedures known in the technique.

As indicated above, the host cell of the invention expresses at least one PMO polypeptide of the invention, which is capable of oxidising cellulose when secreted into the extracellular medium. This host cell is able to secrete this enzyme(s) into the medium along with another/other cellulolytic enzyme(s) produced naturally or recombinantly, thus being useful for the optimisation of the biomass hydrolysis stage in fermentable sugars.

Therefore, another aspect of the invention concerns the use of the host cell of the invention, the polypeptide(s) of the invention, or the composition of the invention, for the enzymatic degradation or hydrolysis of cellulosic biomass.

The term “biomass” refers the biodegradable fraction of products, waste and residues of biological origin from agriculture (including plant substances, such as crop residues, and animal substances), forest industries (such as timber resources) and related industries including fisheries and aquaculture, as well as the biodegradable fraction of industrial and urban waste, such as municipal solid waste or paper residues. In a preferred embodiment, the biomass is straw or the organic fraction of urban solid waste. In a more preferred embodiment, the biomass is vegetable biomass, more preferably selected from the list consisting of: biomass rich in fermentable sugars, such as sugar cane; starch biomass, for example, grains or wheat straw; or maize or maize straw or maize grain or maize fibre; or barley grains or straw; or sorghum grains or straw. Biomass can also include rice, grass, branches, etc. In a more preferred embodiment, the cellulosic biomass employed in this invention comprises corn and/or sugar cane.

The polypeptide of the invention, as well as the host cell or composition of this invention, may be used in the production of monosaccharides, disaccharides and polysaccharides as chemical or fermentation raw materials, from biomass for the production of ethanol, butanol, plastics, alkanes, alkenes, or other products or intermediates.

The host cell of this invention can be used as a source of the polypeptide of the invention, and other cellulolytic enzymes, in a fermentation process with biomass.

The predominant polysaccharide in the primary cell wall of the plant biomass is cellulose, the second most abundant is hemicellulose, and the third, depending on the biomass in question, may be pectin. The secondary cell wall, produced after the cell has stopped growing, also contains polysaccharides and is reinforced by covalently cross-linked polymer lignin with hemicellulose. Cellulose is an anhydrocelobic homopolymer and thus is a linear beta-(1-4)-D-glucan, while hemicellulose includes a variety of compounds, such as xylans, xyloglucans, arabinoxylans, and mannan in complex branched structures with a range of substitutes. Although generally polymorphous, cellulose is found in plant tissue mainly as an insoluble crystalline matrix of parallel glucan chains. Hemicelluloses are normally bound by hydrogen bonds to cellulose, as well as to other hemicelluloses, which helps to stabilise the cell wall matrix. The polypeptides of the invention can be used together with other cellulolytic enzymes to degrade the cellulose component of the biomass substrate.

The degradation or hydrolysis of biomass to fermentable sugars, also known as “saccharification”, by means of the polypeptide of the invention, the host cell of the invention or the composition of the invention, may be followed by a fermentation process in which the fermentable sugars obtained are used in order to finally obtain a bioproduct such as bioethanol.

Thus, another preferred embodiment of this aspect of the invention refers the use of at least one polypeptide of the invention, of the host cell of the invention or of the composition of the invention, for the degradation of biomass in a process for the production of a bioproduct.

The term “bioproduct” or “biobased products” refers to the materials, chemicals and energy derived from renewable biological resources. Examples of these bioproducts are, but are not limited to, hydrocarbon compounds in their different forms, such as aliphatic (saturated, unsaturated, cyclic) or aromatic compounds, such as alkanes, alkenes, alkyls, cyclic forms of these compounds or aromatic hydrocarbons; oxygenated substances such as alcohols (such as ethanol, butanol, sorbitol), ethers, aldehydes, ketones or carboxylic acids; nitrogenous substances such as amines, amides, nitrocompounds or nitriles; halogenated substances such as halides; organic acids (such as lactic acid, acrylic acid, acetic acid, succinic acid, glutamic acid, citric acid or propionic acid). The term “bioproducts” also includes any combination of the compounds described above, compounds derived in addition to the compounds described above by any type of physical, chemical or biological treatment, polymers of the compounds described above, compounds described above replaced by any group or functional element in one or more of their linked and branched forms.

Ethanol can be produced by the enzymatic degradation of biomass and the conversion of saccharides released into ethanol. This type of ethanol is often referred to as bioethanol. It can be used as a fuel additive or extender in mixtures of less than 1% up to 100% (a fuel substitute).

In a more preferred embodiment, the bioproduct is a biofuel. The term “biofuel”, as used herein, refers to a hydrocarbon, or one of its mixtures, that can be used as a fuel and is obtained using fermentable biomass as the starting material. Examples of biofuels include, but are not limited to, ethanol or bioethanol, butanol or biobutanol and biodiesel. In a more preferred embodiment, biofuel is bioethanol.

The term “bioethanol” refers to a alcohol prepared by fermentation from fermentable biomass such as carbohydrates produced in sugar or starch crops such as maize or sugar cane.

In another aspect, this invention refers to a process to produce fermentable sugars from cellulosic biomass, referred to herein as the “first procedure in the invention”, which comprises the following stages:
a) incubation of the cellulosic biomass, preferably pre-treated biomass, with the composition of the invention, with at least one polypeptide of the invention or with the host cell of the invention, and
b) recovering of fermentable sugars obtained after incubation of stage (a).

A biomass pre-treatment procedure is often required to increase enzyme access to their substrates and consequent effective hydrolysis. Pre-treatment uses a variety of techniques, including but not limited to chemical treatment (e.g. ammonium fibre explosion or exposure to a solvent), physical treatment (e.g. steam explosion at high temperatures), mechanical treatment (e.g. crushing or grinding), biological treatment, or any combination thereof, to alter the structure of cellulosic biomass and make cellulose more accessible.

The term “fermentable sugar” as used herein refers to simple sugars (monosaccharides, disaccharides, and short oligosaccharides), such as glucose, xylose, arabinose, galactose, mannose, rhamnose, sucrose, or fructose, etc. A fermentable sugar is any one that can use or ferment a microorganism. Preferably, the fermentable sugars referred to in the invention comprise at least glucose.

Another aspect of this invention refers a process for producing a bioproduct from cellulosic biomass, hereinafter referred to as the “second procedure in the invention”, which comprises the following stages:
a) incubation of the cellulosic biomass, preferably pre-treated biomass, with the composition of the invention, with at least one polypeptide of the invention or with the host cell of the invention,
b) fermenting of the fermentable sugars obtained after incubation of stage (a) with at least one fermenting micro-organism, and
c) recovering the bioproduct obtained after fermentation of stage (b).

The term “fermenting” or “fermentation” as used herein refers to a biological transformation process produced by the activity of some microorganisms in which sugars such as glucose, fructose, and sucrose are converted into ethanol. The microorganisms used in this way are fermenting microorganisms that have fermentation capacity, such as yeasts of the genera Saccharomyces, Pichia or Kluyveromyces, preferably Saccharomyces cerevisiae, both natural and genetically modified strains for the conversion of pentoses.

The term “recovery” as used herein refers to the collection of fermentable sugars obtained after the incubation of stage (a) of the first procedure of the invention or of the bioproduct obtained after the fermentation of stage (b) of the second procedure of the invention. Recovery may be performed by any procedure known in the technique, including mechanics or manuals.

In some embodiments, the first and/or second procedure of the invention comprises, preferably, a process of pre-treatment of the biomass before stage (a). In general, a pre-treatment process will result in the components of the cellulosic material being more accessible for later stages or more digestible by enzymes after treatment in the absence of hydrolysis. Pre-treatment can be chemical, physical, mechanical or biological pre-treatment, or any mixture thereof. Preferably, the pre-treatment of the biomass to which this invention refers is done by steam explosion.

Before (i.e. in stage (a)) and/or simultaneously with the fermentation of stage (b) of the second method of invention, biomass, preferably pre-treated biomass, is hydrolysed to degrade cellulose and hemicellulose into sugars and/or oligosaccharides. The solid content during hydrolysis can be, but not be limited to, between 10-30% of the total weight, preferably between 15-25% of the total weight, more preferably between 18-22% of the total weight. Hydrolysis is performed as a process in which biomass, preferably pre-treated biomass, is incubated with at least one polypeptide of the invention, with the host cell of the invention or with the composition of the invention and thus forming the hydrolysis solution. The correct process time, temperature and pH conditions can easily be determined by a technical expert. Preferably, this hydrolysis is carried out at a temperature of between 25° C. and 60° C., preferably between 40° C. and 60° C., specifically around 50° C. The process is preferably carried out at a pH in the range 4-6, preferably pH 4.5-5.5, especially around pH 5.2. Preferably, hydrolysis is performed between 12 and 144 hours, preferably between 16 and 120 hours, more preferably between 24 and 96 hours, even more preferably between 32 and 72 hours.

Hydrolysis (stage (a)) and fermentation (stage (b) of the second method of the invention) can be performed simultaneously (SSF process) or sequentially (SHF process). According to the invention, hydrolysed biomass, and preferably pre-treated, is fermented by at least one fermenting microorganism capable of fermenting fermentable sugars, such as glucose, xylose, mannose and galactose, directly or indirectly in the desired fermentation product. Fermentation takes place preferably between 8 and 96 hours, preferably between 12 and 72 hours, more preferably between 24 and 48 hours. In another preferred embodiment, fermentation takes place at a temperature of between 20° C. and 40° C., preferably 26° C. to 34° C., particularly around 32° C. In another preferred embodiment, the pH is 3 to 6 units, preferably 4 to 5. A yeast of the Saccharomyces cerevisiae species is preferred for ethanolic fermentation, preferably strains that are resistant to high levels of ethanol, up to, for example, 5 or 7% vol. of ethanol or more, such as 10-15% vol. of ethanol.

In a preferred embodiment of the second process of the invention, the bioproduct is biofuel, more preferably bioethanol.

Throughout the description and the claims, the word “comprises”, “consists” and its variants are not intended to exclude other technical characteristics, additives, components or steps. For experts in the art, other objects, advantages and characteristics of the invention will emerge partly from the description and partly from the practice of the invention. The following examples and figures are provided by way of illustration, and are not intended to be exhaustive of this invention.

### DESCRIPTION OF THE FIGURES

FIG. 1. Analysis of glucose release from pre-treated corn biomass (PCS) subjected to different cellulolytic enzymatic compositions obtained from a strain of M. thermophila control (C) and strains of M. thermophila transformed with expression vectors codifying for each of the proteins of the invention.

FIG. 2. Analysis of glucose release from pre-treated sugar cane biomass (PSCS) subjected to different cellulolytic enzymatic compositions obtained from a strain of M. thermophila control (C) and from strains of M. thermophila transformed with expression vectors codifying for each of the proteins of the invention.

FIG. 3. Analysis of glucose release from pre-treated corn biomass (PCS) subjected to different cellulolytic enzymatic compositions obtained from a strain of M. thermophila control (C) and from strains of M. thermophila transformed with expression vectors generated to code for different signal peptide combinations (SPWT, native signal peptide; SPGA, signal peptide of glucoamylase A; SPPMO, signal peptide of PMO-06230) and mature protein for proteins of SEQ ID NO: 52, SEQ ID NO: 59 and SEQ ID NO: 65 of the invention.

### EXAMPLES

### Example 1. Construct of the Codifying Expression Vectors for the Different Polysaccharide Monooxygenases of the Invention Together with the Different Signal Peptides, for their Expression in M. thermophila C1

The genes of different polysaccharide monooxygenases listed in Table 1 were used to overexpress enzymes in the host cell M. thermophila C1 and to test the hydrolysis yields with the resulting enzymatic cocktails (secreted by the transformed cells).

Genes were synthesised in vitro after optimisation to eliminate restriction sites of the most common enzymes without altering the encoded amino acid sequence. The resulting genes and deduced sequences are referenced according to Table 1.

Using the SignalP tool (Petersen et al., 2011, Signal IP 4.0, Nature Methods, 8:785-786), signal peptides of all proteins were identified. The sequences of the mature proteins are referenced according to Table 1.

Prior to in vitro synthesis, the signal peptide of the native protein was replaced to generate variants with the signal peptide of Aspergillus niger glucoamylase A (glaA, Uniprot:A2QHE1) or with the signal peptide of M. thermophila's own polysaccharide monooxygenase 06230 (Uniprot:G2QCJ3).

The cloning and expression of each gene followed a procedure similar to that described in the patent (PCT/ES2013/070318).

### Example 2. Cocktail Effect Produced by Transforming Strains on Glucose Release During Enzymatic Hydrolysis of Different Lignocellulosic Substrates

The release of fermentable sugars by cocktails produced by the parental strain of M. thermophila C1 (control) and by selected clones resulting from transformation with expression vectors containing coding sequences of enzymes bound to the Glucoamylase A signal peptide was compared by enzymatic hydrolysis. Pre-treated Corn Stover (PCS) or Pre-treated Sugar Cane Straw (PSCS) biomass was used as the substrate. Biomass pre-treatment was performed by steam explosion in the presence of dilute acid (Nguyen et al., 1998, Appl. Biochem. Biotechnol. 70-72; Alcantara et al. 2016, Biotechnology for Biofuels. 9:207), and the compositional analysis of the resulting biomass was performed according to the procedures described by NREL in “Standard Biomass Analytical Procedures” (http://www.nrel.gov/biomass/analytical_procedures.htmL). In order to be used in hydrolysis, the biomass was previously neutralised at a pH of 6.5. For the enzymatic hydrolysis process, 100 ml ISO vials were used with 20 g of the reaction mixture at 20% (w/w) of total solids and supplemented with 8 mg of protein per g of glucan from the cocktail coming from the strains in question. The vials with the mixture were incubated for 72 hrs at 50° C. with 150 rpm agitation in a 25 mm diameter orbital incubator (Infors HT). At the end of the process, the glucose content in the slurry samples was analysed by HPLC (Agilent Technologies, 1200 Series) using a refractive index (DIR) detector and an Aminex HPX-87 H column.

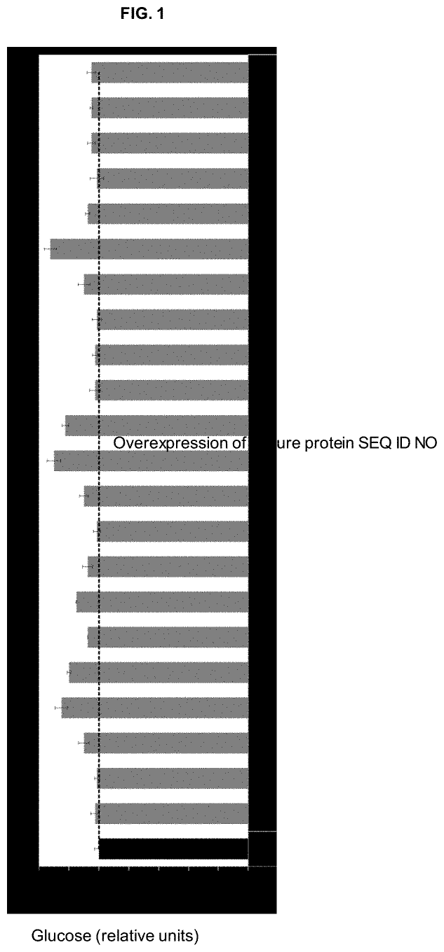

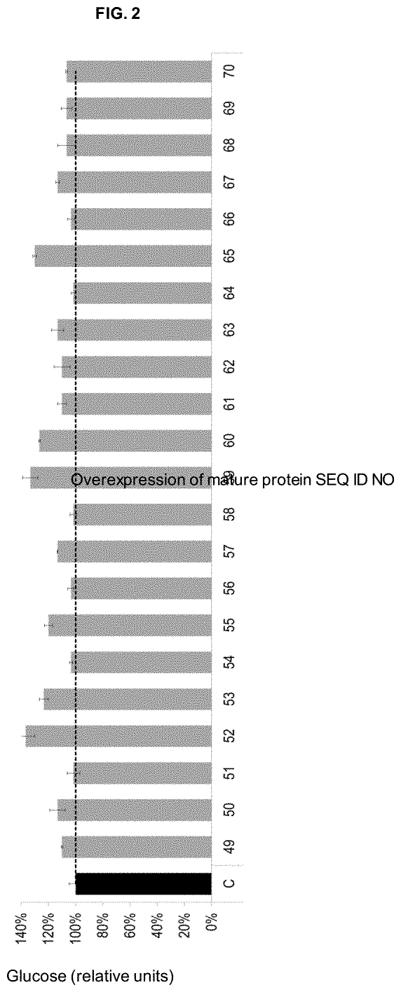

The results obtained with PCS are shown in FIG. 1 and those obtained with PSCS in FIG. 2, where it can be seen that all enzymes overexpressed with the glucoamylase signal peptide release more glucose than the cocktail produced by the control strain, the enzymes being of SEQ ID NO: 52, SEQ ID NO: 53, SEQ ID NO: 59, SEQ ID NO: 60 and SEQ ID NO: 65 the highest glucose release yields provided.

### Example 3. Effect of the Different Signal Peptides on the Performance of Cocktails Overexpressing the Polysaccharide Monooxygenases of the Invention

In this example was compared the cocktail yields obtained by overexpressing 3 polysaccharide monooxygenases (SEQ. ID NOs: 52, 59 and 65) fused to the different signal peptides of this invention: its native or original signal peptide (SPWT), that of A. niger's glucoamylase A (SPGA) and that of M. thermophila's PMO-06230 (SPPMO). The enzymatic cocktail obtained from the parental strain of untransformed M. thermophila C1 was used as a control.

Enzymatic hydrolysis and analysis of the results were performed as explained in the previous example using Pre-treated Corn Stover (PCS) biomass.

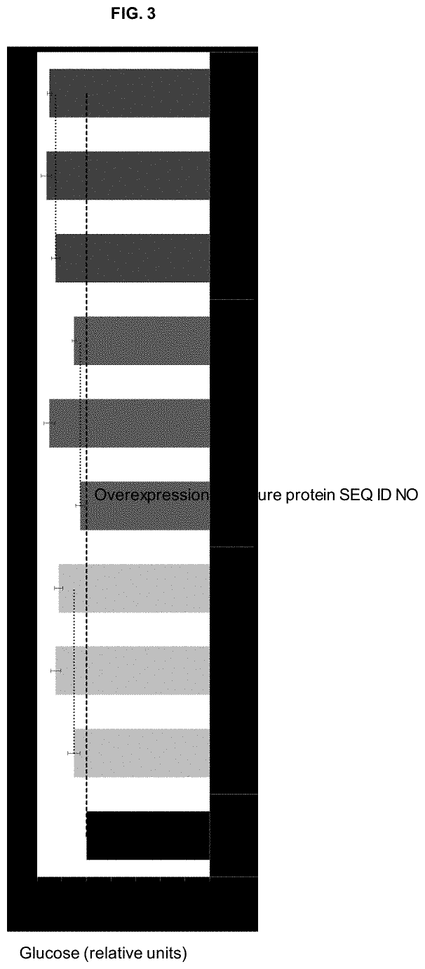

The results obtained, as shown in FIG. 3, show that the increase in sugar release also depends on the signal peptide used (and therefore on the level of overexpression obtained) with each of the enzymes tested, although they all surpassed their parental strain. The signal peptide that provided the highest yields in glucose release was SPGA, followed by SPPMO and finally SPWT.

TABLE 1
Identification of the DNA and protein sequences corresponding
to each of the PMOs of the invention.

| | Protein sequence of the | Protein sequence of the |
|---|---|---|
| Nucleic acid | immature enzyme | mature enzyme |
| SEQ. ID NO. | SEQ. ID NO. | SEQ. ID NO. |
| 1 | 23 | 49 |
| 2 | 24 | 50 |
| 3 | 25 | 51 |
| 4 | 26 | 52 |
| 5 | 27 | 53 |
| 6 | 28 | 54 |
| 7 | 29 | 55 |
| 8 | 30 | 56 |
| 9 | 31 | 57 |
| 10 | 32 | 58 |
| 11 | 33 | 59 |
| 12 | 34 | 60 |
| 13 | 35 | 61 |
| 14 | 36 | 62 |
| 15 | 37 | 63 |
| 16 | 38 | 64 |
| 17 | 39 | 65 |
| 18 | 40 | 66 |
| 19 | 41 | 67 |
| 20 | 42 | 68 |
| 21 | 43 | 69 |
| 22 | 44 | 70 |

1. An isolated polypeptide with polysaccharide monooxygenase activity comprising an amino acid sequence with at least 80% identity with the amino acid sequence SEQ ID NO: 65.

2. The polypeptide according to claim 1, comprising an amino acid sequence having at least 90% identity with the amino acid sequence SEQ ID NO: 65.

3. The polypeptide according to claim 2, comprising the amino acid sequence SEQ ID NO: 65.

4. The polypeptide according to claim 1, which further comprises attached to its N-terminal end a signal peptide, preferably the signal peptide with SEQ ID NO: 46.

5. The polypeptide according to claim 3, consisting of the amino acid sequence SEQ ID NO: 65.

6. The polypeptide according to claim 3, consisting of the amino acid sequence SEQ ID NO: 39.

7. An isolated polynucleotide encoding the polypeptide according to claim 1.

8. A gene construct comprising the polynucleotide according to claim 7.

9. The gene construct according to claim 8, wherein the gene construct is an expression vector.

10. A host cell or a gene construct comprising the polynucleotide according to claim 7.

11. The host cell according to claim 10, wherein such cell is Myceliophthora thermophila C1.

12. An enzymatic composition comprising the polypeptide according to claim 1.

13. The enzymatic composition according to claim 12, which further comprises another cellulolytic enzyme.

14. The enzymatic composition according to claim 13, wherein the cellulolytic enzyme is selected from the list consisting of: endoglucanase, beta-glucosidase, celobiohydrolase, celobiose dehydrogenase, beta-xylosidase, alpha-xylosidase, endoxylanase, endoxyloglucanase, polysaccharide monooxygenase or any combination thereof.

15. The enzymatic composition according to claim 12, wherein the composition is an enzymatic mixture expressed by a cell comprising an isolated polynucleotide or a gene construct comprising the polynucleotide; wherein the polynucleotide encodes an isolated polypeptide with polysaccharide monooxygenase activity comprising an amino acid sequence with at least 80% identity with the amino acid sequence SEQ ID NO: 65.

16. (canceled)

17. (canceled)

18. (canceled)

19. (canceled)

20. A process for producing fermentable sugars from cellulosic biomass comprising the following stages:
a) incubation of cellulosic biomass with the composition according to claim 12, and
b) recovering the fermentable sugars obtained after the incubation of stage (a).

21. A process for producing a bioproduct from cellulosic biomass comprising the following stages:
a) incubation of cellulosic biomass with the composition according to claim 12,
b) fermenting the fermentable sugars obtained after the incubation of stage (a) with at least one fermenting microorganism, and
c) recovering the bioproduct obtained after the fermentation of stage (b).

22. The process according to claim 21, wherein the bioproduct is a biofuel.

23. The process according to claim 22, wherein the biofuel is bioethanol.


SEQUENCE LISTING (70 sequences)

SEQ ID NO: 1 - DNA, 1147 bp, Byssochlamys spectabilis
  sig_peptide (1)..(63)
  Intron (102)..(153)
atgccgttctctaagatagctatcctcgctaccattcttggtaccgcatctctggtttcaggacatggatatgtccaagggattgttgccgatggaaaatagtatgttctgtagctatacctgacagtgagcgggtgcttactatctttacagctatggcggatatatcgtgacgcagtatccgtacaccaacgaccctcccgagctcgtcgcctggtctaccgaagcaaccgatcttggatttgtggacggaagtggctatgcttcaggtgatatcatctgccacaagagcgcacagcctgccgccctggctgctgaggtaccagctggagggaaagtagagcttcaatggaccgaatggcctgagagccaccacggtcctgtgcttgattaccttgctccttgcaacgggaattgcgccgacgttgacaagaccgccctggaattcttcaagattgaccagggtggtttggtcgatgacagcagccctccaggcacctgggcctctgatcaattgatttccaacaacaactcctggaccgtcactattccctccacaatcgccccaggtaattacgttctgaggcacgaaatcattgcccttcactccgccggaaacaaggatggagctcagaattatcctcagtgtatcaacataaaagtcactggttccggtaccgattctccttctggcactctcggcacgtccctttataaggacactgatcctggcattctggtcaacatttatcaaacgctgtccgattattccattccaggccctgcgctatatgaaggtggcagtagctctggcacatcctctggctctggaacttcgacggccacttcgacctctgtggcatcttccacctcagttaagccttccagtattagcactccttcctcagttcaagtggctatcccatctgccaaaacctcagctgttcctgctacgacgcaggcacctaccacagctactcctgcggtcacgattccaactgaaagcgccttgaccgattacctcaacagtatgagtgcagatgaggtcctgaatctcgttcgtggtactatgtcctggttgttcacgaacaagaatcacgctcgagatttatctgctagttctgagcttactgatcgccccatgtaa

SEQ ID NO: 2 - DNA, 750 bp, Penicillium oxalicum
  sig_peptide (1)..(63)
atggtatcttcaaaagccaccaccctatctgccatgttggcatgtgtctctatggtcgcaggccatggtttcgtttccagcattatggcaggtggcaaaaactacactggctatctcgccaactcctacccatacatgagcaatccaccccagagcgtcggctgggccaccacagctaccgatctaggctttgaagacggcaccgaatatcaatcggccaacatcatctgccaccgagatggcaagaatgccgcactctctgcacccgtcaccgcaggctccaaggtggaaatccaatggacctcatggccagacagccaccatggtcccgtcatcacataccttgcctcctgtggtggagactgcagcacagtcgacaagaccacgctcaagttcttcaagatcgatgaggccggtctgattgatgactctaaccctcccggaacgtgggccacggatcagctcattgcagctggaaaccgatggacggtgactatccccaagagcattgcatcggggaactatgtcatgcgtcacgagatcatcgctcttcattcttcgggttccaaggatggtgcacagaactatccccagtgtttgaatctgcaggtcaccggtggagggagcgctaagcctcagggtactttgggtgaggctctgtacaaggacactgatcctggcattttgatcaatatctaccagtccttgaagagctacgtgattcccggtccagctctgtacagcgcgtga

SEQ ID NO: 3 - DNA, 1032 bp, Aspergillus clavatus
  sig_peptide (1)..(57)
atgtctgtcactaagatcgctggtatccttctcggctccgccgccatggtcgctggtcacggtttcgtgaccggtgccgttgtcgatggcacataccacacgggttatcttgtcaacaactacccctactcgagcaaccctcccaaaaccattggctggtctgagaccgccaccgacctcggctttgtcgacggcactggctacgccagcggcgacatcatttgccacaagaacgctaagcctggagccctctctgcggacatcaaggctggtggaaaggtggagttccaatggacccagtggcccgagtctcaccacggccctgtcatcacttatatggccaactgcaatggcgactgcgcctcggttgacaagtctcagctggagttcttcaagattgaggagaagggtctcatcggctccggacactgggcctcggatgacctgattgccaacaacaacagctggaccgtgaccgtccccagcagcctggccgctggcaactatgtcatgcgtcacgagatcattggcctccactcggctggcaacgcgaacggggcccagaactacccccagtgcatcaacctcaaggttaccggtggtggcagcgacaagcctgttggcaccctgggcaccaagctctacaaggccaccgaccccggtatccttgtcaacatctacggcagcctgacctcctacacgatccccggccctgctctctggaccggtgcttcttctggcggctccgatgacaacggttccactcccgcttccactcccactcccaccgccgccccctcctccactgctgcccccagcgtgaccatcaccagcacccctggacagaagacccagacccctgtcgccacccccgccccgtctacccccagctcgaccccgggtcttcccaccgacggcaacctgaccgactacttcaagtccctgagccctgagcagttcctcagcgtcctcaaggagaccttttcctggcttgttaccgagaaggttcacgctcgctccctctctgcttag

SEQ ID NO: 4 - DNA, 1178 bp, Aspergillus fumigatus
  sig_peptide (1)..(57)
  Intron (325)..(380)
atgtctgtccctaagattgcagctgctcttctcagctcggccgctctggtcgctggtcacggttttgtgacgggtgccgttgttgatggcaaatactacactggataccttgtcaaccaatacccttacatgagcagccctcctgacagcatcggttggtctgaaaccgctaccgatttgggtttcgtcgacggcagcggctactctagcggcgacatcatctgccacaaggatgctaagaatggtgccatctctgccgagatcaaggccggtggaaaggttgaattccaatggactgaatggcctgaatctcaccacggaccggtatgtcttcgtaagacatccatggaacaattggttagggtattaacttcgtaaaggtcattacctacatggccaactgcaatggtgattgcgcctcagttgacaagactactctggagttcttcaagattgatgagagtggtctgatcagtgactccaatgttcctggcacctgggcctctgacaacctgatcgccaacaacaacagctggaccgtgactgttcccagctccatcgccgctggtaactatgtcatgcgtcacgaaattattgccctccactccgctggcaaccagaatggtgcccagaactaccctcagtgcatcaaccttaaggtcaccggtggtggcagcgacaagcctgcaggtaccctcggcactgcgctctacaagaacactgatgctggcatcctggtcaacatctaccagagcctgagctcctatgaaattcctggtcccgctctctactctggcgcttcttctggcagctccaacaacggtggttccgcctcgtctagcgccactgctccttctgccaccatcactcagccctctactgcagtcccgactagctctgcgactgcttaccagccctccactaccaccgaggctgtcacagtcaccagcattcctgcccagcagagctacgtccaggctcctaccgccacccctagctccactgccggcagctccggctccggctccggttctagctccagtggcactcttcccagcagcagcaacctcactgagtacttcaactccctcagcgccgaagagttcctgaaggtccttaagcagaccttctcttggctggttacggagaaggtgcacgctcgtgatctctctgcttag

SEQ ID NO: 5 - DNA, 1209 bp, Aspergillus niger
  sig_peptide (1)..(57)
atgtctgtcgctaagattgctggtgtcgtcctcggctcggccgcccttgttgctggccacggttatgtgtctggtgctgtcatcgacggcgagtactacggaggatacatcgtctcctcctacccctatgagagtgaccctccggagaccattgcctggtctaccacagccactgaccttggcttcgtcgacggctctgaatactcggaccctgacatcatctgccacaagagcgctaagcctggtgctatctccgctgacgtcaaggccggtggtactgttgagcttcagtggactgactggccttccagccaccacggtcccgtcctgacttaccttgccaactgcaacggtgactgctccgatgtcaccaagactgaccttgagttcttcaagatcgacgagagcggcctcattagcgacactgaagtccccggaacctgggccactgacaacttgatctccaacaacaacagctggactgtgaccattccctcgaccctcgaggctggtaactacgtcctgcgccacgagatcattgccctgcactccgctgagaacaaggatggcgcccagaactacccccagtgtctcaacctgaaggttaccggcagtggtagctccacctactccggcaccaagggtgaagccctctacaaggacaccgacccgggtatcctggtcaacatctacgagaccctgagctcctacgacatccccggtcctgctatgtacaacgccacttcctccagcagctcttctggcagctcctccggtgcctcttctaccagctccgctgccgctgccgccgccactacttccagtgcctcgacttccagcgcttccacccccaccaccagcgctgccgctgcgactagctccgccgtaagcgccaccaccactgctgctgctgccgttgtcaccgacgttgaaaccaccgttcagactgcggtcgccactgcttaccagacttcgactgacgttgtgcaggtcactgtcactggcagcgccccccagcagacccacatccaggtcgccagctccagcgccgctgcttccacccccagcagctccagtgtcgccgtctccagcgacaacctcaccagctacttcagctctctgagcgccgaccagttcctcagcgtcctgaaggagaccttctcttggctggtcactgagaagaagcacgcccgtgacctcaacgcctaa

SEQ ID NO: 6 - DNA, 1209 bp, Aspergillus niger
  sig_peptide (1)..(57)
  Intron (732)..(878)
atgtctgtcgctaagattgctggtgtcgtcctcggctcggccgcccttgttgctggccacggttatgtgtctggtgctgtcatcgacggcgagtactacggaggatacatcgtctcctcctacgcctatgagagtgaccctccggagaccattgcctggtctaccacagccactgaccttggcttcgtcgacggctctgaatactcggaccctgacatcatctgccacaagagcgctaagcctggtgctatctccgctgacgtcaaggccggtggtactgttgagcttcagtggactgactggccttccagccaccacggtcccgtcctgacttaccttgccaactgcaacggtgactgctccgatgtcaccaagactgatcttgagttcttcaagatcgacgagagcggcctcatcagcgacactgaagtccccggaacctgggccactgacaacttgatctccaacaacaacagctggactgtgaccattccctcgaccctcgaggctggtaactacgtcctgcgccacgagatcattgccctgcactccgctgagaacaaggatggcgcccagaactaccctcaatgtctcaacctgaaggttaccggcagtggtagctccacctactccggcaccaagggtgaagccctctacaaggacaccgacccgggtatcctggtcaacatctacgagaccctgagctcctacgacatccccggtcctgctatgtacaacgccacttcctccagcagctcttccggcagctcctccggtgcctcttctaccagctccgctgccgctgccgccgccactacttccagtgcctcgacttccagcgcttccacccccaccaccagcgctgccgctgcgactagctccgccgtaagcgccaccaccactgctgctgctgccgttgtcaccgacgttgaaaccaccgttcagactgcggtcgccactgcttaccagacttcgactgacgttgtgcaggtcactgtcactggcagcgccccccagcagacccacatccaggtcgccagctccagcgccgctgcttccacccccagcagctccagtgtcgccgtctccagcgacaacctcaccagctacttcagctctctgagcgccgaccagttcctcagcgtcctgaaggagaccttctcttggctggtcactgagaagaagcacgcccgtgacctcaacgcctaa

SEQ ID NO: 7 - DNA, 1035 bp, Aspergillus ruber
  sig_peptide (1)..(57)
atgtctgtcaccaagatcactggtcttcttctcggctcggccgctatggtcgctggtcacggttatgtctccggtgccgttgtggatggccaatactacggaggatacatcgttacctcctacgcttacaccgacagccccccagaaaccattggatggtcgaccgaagctactgatctgggctttgtctctcccagctccttctctgacccggatattatctgtcacaagagtgcgcagcccggtgccatctctgcttcggtggcagctggtaaggatgttgagcttcagtggactgaatggccggagagtcaccatggtcctgtcatcacctacctggccaactgcaacggtgactgctccaaggtcgacaagacctcgctcgagttcttcaagatcgaccagaagggtctcgtcgacgacagcaacgtccctggcacatgggctagcgacaatctgatttccaacaacaacagctacaccgtcactattcccagcgacattgctgccggtaactacgtcctgcgtcatgaaattattgccctgcactccgctggaaacgaggacggtgcccagagctaccctcaatgtgtcaacctcaaggttactggtggcggcagcgcatctccctcgggtactcttggtactaagctctacagcgaggacgacccgggtatcctcgtcaacatttaccagcagctcgactcctacgaaatccctggccctgctctgtactctggcgcttcttcgtcctccaactctggctcttcttccagcactgcttcggcttctactactgccacttctgccactacttcgtctgcctcgtccacccaagcttccgctacccctgcctcccaagccaaggcccaagtctctagctctactcccagcgcttcgtccgttgcgacttctggtagcttgtccgactacttcagctccctgagcgctgaggagtttctcaacgttatcagcgagactctgtcctggttggttactgacaagatccacgcccgtgacatctcgaccgcataa

SEQ ID NO: 8 - DNA, 1209 bp, Aspergillus terreus
  sig_peptide (1)..(57)
atgtctgtcgctaagattgctggcgttgtcctcggctctgcggcccttgtcgctggccatggttacgttacgggtgccgtcgtcgatggcaagtactatgctggctacgatgtcacctctgccccttactctagcgaccctccggccaccatcggctggtcgaccaccgccaccgacctcggcttcgttgacggcaccgagtactccgagcctgacatcatctgccacaaggacgctaagcctggctctctctctgctgaaattactgccggtggcaaggtcgagttgcagtggaccgaatggcccgagagccaccacggccctgtcattacctacctggccaactgcaacggcgactgctcctcggtcgacaagacttcgctggagttcttcaagattgatcagaagggtctcattgagggcaacacctgggctagtgaccagctgatcagcaacaacaacagttggactgtcaccatccccagcagcattgcttctggcaactatgtcctgcgccacgagatcattggccttcactccgctggcaacaaggatggcgcccagaactaccctcagtgcatgaacctcaaggttactggtggcggcagcgacaagccggctggcacccttggtaccgctctgtacaaggacactgaccctggtatcctcgtcaacatctaccagactctgtcctcgtacgtgatccccggtcctgccctctactctggcagctcatccggctcgggttcgggctcagctagctccagcgctgctgctactccttcctccagcagtgttccctccagcaccagcattgccgttcccaccagctctgctcctgcccccacctccgccgctccttcggctcaaaccagcactgctactgcctaccagactgccaccactattgagagtgtcaccgttaccgccgaagcgccccagcagaccaaggtgcaggaccctagctccggctctagctctggttccaactctggctccagctccggttctagctctggatccagcactggctctagctccggctccagcactggcactggctcctccagtgttcccactcctagtggtagcagcagcctgtctgactacttcaactccctgagtgccgagcagttcctgaaccttctccgccagactctttcttggctgatcaccgacaagaagcacgcccgtgacctctctgcctag

SEQ ID NO: 9 - DNA, 1160 bp, Neosartorya fischeri
  sig_peptide (1)..(57)
atgtctgtctctaagattgcagctgttcttctcagctcggccgctctggttgctggtcacggattcgtgtcgggtgccgttgttgatggcaaatactacactggatacctcgtcaaccaatacccttacatgagcagccctcctgagagcatcggttggtctgaaaccgctaccgatctgggtttcgtcgacggtagcggctactctagcggcgacatcatctgccacaagagtgctaagaatggtgccatctctgccgagatcaaggccggtggaaaggttgaattccaatggactgaatggcctgaatctcaccacggaccggtatgtcttcgtaagacatccatggaataatcggttagggtattaacttagtacaggtcattacctacatggccaactgcaatggtgattgcgcctcagttgacaagaccagtctggaattcttcaagattgatgagagtggtctgatcagtgactctaatgttcctggcacctgggcctccgacaacctaatcgccaacaacaacagctggacggtgactgttcccagctccatcgccgctggtaactatgtcatgcgtcacgaaatcattgccctccactccgctggaaaccagaacggtgcccagaactaccctcagtgcatcaaccttaaggtcaccggtggtggcagcgacaagcctgcaggtaccctcggcactgcgctctacaagaacactgacgccggcatccttgtcaatatctaccagagcctgagctcctatgacattcctggtcccgctctctactctggcgcttcttctggcagctccaacagcggtggttccgcctcgtctagcgccgctgctccttctgccaccatcactcagacctctactgcagtccagactagctctgcgactgtttaccagccctccactaccaccgaggctgtcaccgtcaccagcactcctgcccagcagagctacgtccaggctcctaccgccacccctagctccactgccggcagctccagctccagcggcactcttcccagcggcagcagcctcaccgagtacttcaactccctcagcgccgaggagttcctgaaggtccttaaggagaccttctcttggctggttacggagaaggtgcacgctcgtgatctctctgcctag

SEQ ID NO: 10 - DNA, 1203 bp, Aspergillus kawachii
  sig_peptide (1)..(57)
atgtctgtcgctaagattgctggtgttgtcctcggttcggccgcccttgttgctggccacggttatgtgtctggtgctgtcgttgacggccagtactacggaggatacatcgtctcctcctacgcctatgagagtgaccctccggagaccattgcctggtctaccgaagccactgaccttggctttgtcgacggttctgaatacgccgagcctgacatcatctgccacaagagcgctaagcctggtgctatctccgctgacgtcaaggctggtggtactgtcgagcttcagtggactgactggccctccagccaccacggtcccgtcctgacttaccttgccaactgcaacggtgactgctccgatgtcaccaagactgaccttgagttcttcaagatcgacgagagcggccttatcagcgacactgaagtccctggaacctgggccaccgacaacttgatctccaacaacaacagctggactgtgaccatcccctcgaccctcgagtctggtaactacgtcctgcgtcacgagatcattgccctgcactccgctggaaacaaggatggcgcccagaactaccctcagtgcctcaacctgaaggttaccggtagcggtagctccacctactcaggcaccaagggtgaagccctctacaaggacaccgacccgggtatcctggtcaacatctaccagagcctgagctcctacgacatccccggccctgctatgtacaacgccacttcttccggcagctcctccggcagctcctccagtgcctcttctacgagctccgctgccgctgccgctgctactacttccagcgcttccagctccaccaccagcgctgctgctgcgactagctccgccgtaagcgccaccaccaccagcgctgctgctgctgccgttgtcaccgacgttgaaaccaccgtccagactgcggtcgccactgcttaccagacttcgactgacgttgtgcaggtcactgtcactggcagcgccccccagcaaacccacatccaggttgccagctccagcgccgccgctgttgctacccccagcagctccagcgtcgccgtctccagcgacaacctcaccagctacttcagctctctgagcgccgaccagttcctcagtgtcctgaaggagaccttctcctggctggtcaccgagaagaagcacgcccgtgacctcaacgcctaa

SEQ ID NO: 11 - DNA, 1357 bp, Aspergillus nidulans
  sig_peptide (1)..(57)
  Intron (320)..(371)
atgtctgtcgctagaactgctggttttgccctcgcctccgcggccatcgtcgccggtcacggttatgtcacgggaatcgtcgctgacggcacatactacggcggttacctcgttaaccagtacccttacagcaacgacccccctgcagttgttggatgggctgaggatgctaccgacctgggtttcgttgatggctccggctacacctctggcgatatcatctgccacaaggacgctaccaacgcccaggcttctgccaccgtcgctgctggtggcaccgttgagctgcagtggactgagtggcctgaatcccaccacggtgagtacaaactttagctctctgaacatcgcattcacttatacatgcaaaggtcccgtcatcgactacattgccagctgcaacggtgactgcacaaccgtcgacaagaccaccctcgaatgggtcaagattagcgagagcggtctcgttgacggcagctctgcccctggcacctgggcctctgacaacctgatctccaacaacaacagctggaccgtgaccatccccagcagcctcgccgccggtggctacgtcctccgccacgagatcatcgccctccactccgccggcaacgagaatggcgcccagaactacccccagtgtgtcaacctcgaggtcactggcggtggcagcgcctctccctctggtaccgtcggtactgagctttacacccccactgaccctggtatcctggtcaacatctacacctccctcgactcctacaccatccccggccctgctctctgggacggtgcctcttccagcggcggaaacagcggcagtggtagcgcctcctccagcgccgccgccacctccacccctaccactcccagcgtaagcgtccctgtcatccccaccgctagctctggtgcctcctctacccctctcgtgcccacccccagcgcgcctgctgtgaccccttcggtccctgccggcaaccaggcccctcagcccacttacacctccacctacattgagactgagactttgcctggtcagaccgttacctccaccacgaccgagtacgcttccgagcctacccagcccgctgtcgagacccaggttgctcagccctctgagaccgaggctgccacatccacttccactgtcaccgagactgcctctgccaccgccgccccgactggctcctctggctctagctctggatctggctccagcagcaccgagctccccaccgacagctccagcctctccgactacttctcctccctgagcgcagaggagttcctcaaccttctgaaggagacccttaagtggttggtcaccgacaaggtccacgctcgttccctccactaa

SEQ ID NO: 12 - DNA, 806 bp, Aspergillus oryzae
  sig_peptide (1)..(63)
  Intron (102)..(154)
atggctatgtccaagatcgtgtcgcttactggcctcctggcctctgcctctctcgtcgccggccacggttatgtgtctggagtcgtcattgatggtcaatagtatgattctaaattcgcaaaaaagtcattataaaggctaacacccaccatagctatggtggataccttgttgacaagtacgcctactcggacaatgccccagataccattggctggaccacctctgccaccgaccttggattcgtggacggcactggataccagtcccccgatatcatctgtcacaaggacggagcgccaggtgctttgactgctgaagttgctgctggtggcaaaattgaactgcagtggactgagtggcctgaaagtcaccacggccctgtcctcaattacctcgcaccctgcggcggcgagtgctccgcggtggacaagacaactttagagttcttcaagatcgaggccaagggcctgattgacggcaccacccccccgggtcagtgggctaccgatgacttgatcagcaacaacaacagctacactgtgaccatcccgacatccatccaagagggcaactatgttctccgccacgagatcatcggccttcactctgccggtcagaaggatggtgctcagaactacccccagtgcatcaatatcaaggtcactggcggcggagacgccacgccggcaggtaccgccggcgaggccctttacaaggacactgacgctggtattctgttcgacatctacagtgacctcagcggcggctaccccatccctggccctgaggttttcagcgcttaa

SEQ ID NO: 13 - DNA, 1113 bp, Aspergillus oryzae
  sig_peptide (1)..(57)
atgtctatcgctaagattgctggtgtcgtcctcggctcggccgctttggtcgccggtcacggttacgtctctggtgccgtcgttgacggacagtactactctggatatgacatgtcctaccactatatgtccaacccccctcaggtcattggatggtcgaccgatgccaccgacctcggattcgttgacggtagctcatatgccgatgccgacattatctgccacaagaacgccaaaaacggcgctatctctgctgagatcgctgctggcaagcaggtcgagctgcagtggactgcctggcccgagagtcacaagggtcctgtcatcacctacctcgccaactgcaacggcgactgcgccaccgtcgacaagacccagctcgagttcttcaagatcgatgagaagggcctcatcagcggcagtgacaacacctgggcctccgacaatctgatctccagcaacaacagctggaccgtgaccatccccagcagcattgctgccggcaactatgtcatgcgtcacgaaatcattgcccttcactccgctggtaacaaggatggtgctcagaactacccccagtgtctcaacttcaaggttaccggcggtggtagcgacaagcccgagggtaccctcggtactgctctctacaaggacaccgaccctggtattgagatcaacatctaccagaccctgtcctcctacaccatccccggccctgctctctacactggcagctcttccagcggctcctccggctccggcagctccagcgccgctccctcccctactgccagtgcttctgcttccgccactgctgcccctgtccagactagcaccgctactgcctaccagacctccactgctgttgccagcgtcactgttactggctctgctcctgctcagacccacgtccaggccaccagctcttcagctgctgcttccactcccaccgctagctctggcgccagcactggctctggctctggctccagctccagcgacctgaccagctacttcaactccttgagcgctgacgagctcctgaacgttatcaagcagactctgtcttggcttgtcaccgacaagatccacgctcgtgacatctctgcctaa

SEQ ID NO: 14 - DNA, 1474 bp, Baudoinia compniacensis
  sig_peptide (1)..(57)
  Intron (162)..(222)
  Intron (973)..(1227)
atgtcagtcatccagtctgcggccatccttgcgtcgctggttgcctccgtcgcagcacacggacatgtccagggcatcgtcgctggtggcacctactacaccggctacgatccttcgttccaatactcccccatcccacccatcgttatcggctggtccgagtaagtccgcggactgccctgttacaagactgttcacgcaacgctaacggcttcccaccagcccacaagacctggacaatggcttcatagcgccgagcaactacaccaatccggacataatctgccaccgcaacgccaccgcggctggcacctccgcccgcatcgccgccggcgacgtcgtcgaaccccagtggacgccctggccgagcagtcaccacggcccggttattgactacctcgccaaatgccctgggtcctgctcagacgtcgataagacgaccctcgagttcttcaagatcgatcaggttggactgatcgacgataccacggagccaggtacttggggctcggatcaactcctcgccaacaacaacagctggaccgttaccatccctccctccatcgcaccgggcaactacgtcttgcgccacgaaatcatcgcgctacactctgcgggcacggcggacggggcgcagaattacccgcagtgcgtcaacctggaaattacggggagcgggactgatactccggctggcacgctggggcagaagctgtatggtgctaatgactctgggattctgatcaacatctatcagagtttgagcacgtatgaaataccgggtccgagtttgtacagtggcgctgtggcgatgagccagactcaggtggctattacgggatatgcgccgcttagttctggtggcgttgcggtatcgattgcttatagttctgccacaacgacgacggccgcggcaggatcttcgtctgtggcgcgttcgactaccgctgcgtctgcgtctgcgtcgaccgcgttagtgggaagctatgcgtggtcgtcgacaagtattcgggtgtctggaacgccatcttcgacagctttaccggcggcatcatcttcgcacgtatctgcgagcaggccaacagcctcggtatcaccaccggcggtcatcaccgtcgtcaccacgctcaacaccaccactaccattagccctactacgactcactcttcccacaccactctcacgaccgccaccgtcccaacctccctcctcgccgccgtctcctccgccgtcgtacccctcgaaagcatcgtctctgccctcccgagcacggcgttgacgcagacactgcccactgtcttaccgactggaacggcggtgtcgagcaacgacacaatcgccggcttgccgcccgcagaggcacctccggcaggcacgacattgcaggatctcttcgcatggctcgaatatatgcttgaggggctagcgagtactggaggacgacatcagagggattttaaagagcggaagtaa

SEQ ID NO: 15 - DNA, 795 bp, Penicillium roqueforti
  sig_peptide (1)..(54)
  Intron (93)..(149)
atgacactttcaaagctatctacgctcctagcctctgcctctcttgttgcaggccacggctatgtctctagcatcgtagctgatggcaagaagtaagtcaatctaagaaacaaagtctccataccaataacaactaaccacattatcagctacactggctacatcgttgacaaatacccctacatggcagacccccctgacagtgtcggctgggcaacaacggcaaccgacctcggttttgaagacggcaccgaataccaagatccgaatataatctgccaccgaaacggagtcaacgcagctttgagcgccaccgtcaccgctggctccagtatcgatctccaatggacgacctggccagactcacatcacggtcctctgataacttacctcgcctcatgcaacggtgactgcagcacagtcgacaagacgacactaaagttcttcaagattgaagaagatggtctcatcgatggtgcaactcctcctggtacatgggctaccgatgatcttattgctgctggaggccagtggactgtgaagatcccccccactcttgctgcgggtaactatgtgatgcgccatgagattatcgctctgcactctgctggccagttgaatggcgctcagaactacccacagtgcttcaatttggaaatcactggcggtggaagtgatgtgccggatggcacgtttggtggggagttgtatatcaatactgatcctgggatcctgttcgatatttattctacgcttacttcgtatattatcccgggaccggctttgtatgcctga

SEQ ID NO: 16 - DNA, 1007 bp, Sclerotinia sclerotiorum
  sig_peptide (1)..(54)
atgtctttctctaagctcaccactttcgccggcctggtcgccacagttgctgcccatggttatgttactggtattgtagctggtggaacttagtaagttaaatcttggagccatgtattcaaacatactgattcaaaaatagtaccaaaggatggctcgtcaactatgcataccaaaacccaatgccagagtcaattggttggtctgaccaaaatctcgaccttggattcactgcaccatccgcctatgccactggggatatcatctgtgctaaaaatggtaccaatgctgctctttcagctaaggttgctgccggtgacaccgttgattttcaatggactgtttggcccgattctcataaaggcccagtcatcacctacttggcaaactgcaacggagactgcagtaccgtcgataaaaccaagcttgaattcttcaagatagacgaaggaggattgatcgatgacaccactgtccctggaacctgggcttctgacaacttgatcaccaataacaatacatggacatccaccattccagccgatctagctcctgggaactacgtcgctcgtcatgaaattatcgctcttcacggtgcgggaagtaatgatggtgctcaaaattaccctcagtgtatcaacttggaagtcactggtagcggtactcttgctccatctggtactctcggtgaaaagctttacactcctaccgatccaggtatcttggtcaatatctatgctaaattgacctacactattcctggtccacctatgtacaacgcaactggagcagcaactggagcagcaactggagccactaccccagccaccggtaaaaccgcctcagcaacctccgccgccgccgctctttccaccccagctgttgtttctacccctccaactccaaccacaatgattacatccgtcgctcctgccgctacccctgctacctctgctgccgcttcatctggtgctagtggtgatgatgacgacacctgtgaagcatga

SEQ ID NO: 17 - DNA, 1449 bp, Penicillium roqueforti
  sig_peptide (1)..(57)
atgtctgtctcaaaaattgcgggtctcctgcttagctcggccgccatggtcgctggtcacggtttcgtgtcgggtgccgttgttgatggcacttaccacgggggcttcatcgtcaatacatacaactacatgaccgaggtgcctgcgaacattggatggagtgaaaaggccaccgacctcggctacatcgacggctctggctacactggctccgacattatttgccacaaggaagccactccgggtgcgatctccgccgaggtcaaggccggtggctctgtcgagctgcagtggactgactggcccgagagccaccacggcccggtcattacctacatggctaactgcaatggcgactgctccgtggtcgacaagacaaccctcaagtttttcaagatcgccgaggccggtctgatcgacgacactaacgtccccggtacatgggcaagtgaccaactcattgctgccaacaacagcgccactgtgactatccccagcaccatcgccgctggtaactatgttctccgtcacgagatcattgccctgcactccgccggcgatgcaaacggcgcccaaaactacccccagtgtatcaacctgaaggtcaccggcggcggcagcgactcccctgacggcgtcctcggcacagcgctctacacgcccaccgatcccggtattttgatcagcatctacagcgcactggagtcctatgtgatccccggccccgctttgtacaccggcgcttcatccggctcttcatcgacctcctcgaccaccgccgcagccactaccgaagccactaccgcgtctgctactgtttctaccaccgcggcccccaccgtggctactaccagtgcctcaaccgtgagtgcctctcctatccagagctcctcttcgcaccacctcacccgcacccgcacccgcactgcccgtcctacgtacagccctactttctcgtctgctcccacgtctgctgtctcaacttccgcccctagtgggccagattatgagcccactgatgctccttccaccctcatcgagaatgcgactgccaccgcttcctctgctcctcaggttactgacgagcctgctacacagaccgccagcaccgccgacagcatcaccatcaccaccaccgccggtggtctgtctgcccagagctccgcagccgtcctcgagtctagcgccacttccactgtcactgccgaggctactgctactgctgcccctaccgccgcagccaccacttccacctctagcaccagctctggatcttcttcctccagctccacctccagctccggatcttcttccactagctccacctctacctccaactcctcgtacctcagctccctgagcgccgagaagctcctcgaggtcatccggtcgaccctgaagtggcttgtctctgacaagaaggtgcacgcccgtgctctagcttactaa

SEQ ID NO: 18 - DNA, 796 bp, Penicillium italicum
  sig_peptide (1)..(54)
atgacactttcaaagctatctatgctcctagcctccgcctctcttgcggcaggccatggctacgtctccagcatcgtagctaatggcaaaaagtgggttaatccaataccaaaaatctccataccaacaagtaactaaccacatcaccagttatactggctacatcgttgacaaatacgcttacatgtcagacccccctgacagtgtcggctgggcaacaaccgcaaccgatctcggattcgaagacggcactgaataccaaaatccgaacataatctgccatcgaggcggagtcaacgcagctttgagtgccactgtcgccgctggctccagcattgatattcaatggaccacctggccagactcgcaccatggtcctctgatcacgtacctcgcctcgtgcaacggtgactgcagcacagtcgacaaaaccacactgaaattcttcaagatcgaagaagatggcctcatccatggcgcaactcctcccggtacgtgggccaccgatgatcttattgcttctgggggccagtggactgtgaggattccttccaccatcgctacgggtaattatgtcatgcgccatgagattatcgctctgcactctgctggccagttgaatggcgctcagaactacccgcagtgtttcaatttgcaggtcactggtggtggaggtaatgcgcctaatggcacgcttggtgagaagttgtatactaatactgatcctgggatcttgttcaatatttactctacgttgacttcttatgtgatcccgggaccggctttgtatgcctga

SEQ ID NO: 19 - DNA, 1119 bp, Aspergillus flavus
  sig_peptide (1)..(60)
atgtctatcgccaagattgctggtatcgtcctcggctcggccgctttggtcgccgctcacggtcacgtctctggtgccgtcgttgacggacagtactaccctggatatgacatatcctaccactacatgcccgatccccctaaggtcattggatggtcgaccgatgctaccgacaatggattcgttgacggtagctcatatgccgatgccgacattatctgccacaagaacgccaagaacggcgctatctctgctgagatcgctgctggcaagcaggtcgagctgcagtggactgcctggcccgagagtcacaagggtcctgtcatcacttacctcgccaactgcaacggcgactgcgccaccgtcgacaagacccagcttgagttcttcaagatcgatgagaagggcctcatcagcggcagtgacaacacttgggccaccgacaacctgatcgccagcaacaacagctggaccgtgaccatccccagcagcattgctgccggcaactatgtcatgcgtcacgaaatcattggccttcactccgctggccagaaggatggtgctcagaactacccccagtgtctcaacttcaaggtcaccggcggtggtagcgacaagcccgagggtaccctcggtactgctctctacaaggacaccgaccctggtatccagatcaacatctaccagactctgacctcttacaccatccccggccctgctctctactctggcagctcttccagcggctcctccggctccggcagctccagcgccgctccctccgctaccgccagtgcttctgcttctgcttctgccactgctgcccctgttcagactagcaccgctactgcctaccagacctcaactgctgttgccagcgtcactgttactggctctgctcccgctcagacccacgtccaggccacgagctcttcggctgctgcttccactcccaccgctagctctggcgccagcactggctctggctctggctccagctccagcgacctgaccggctacttcaactccttgagcgctgacgagctcctgaacgttatcaagcagactctgtcttggcttgtcaccgacaagatccacgcccgtgacatctctgcctaa

SEQ ID NO: 20 - DNA, 798 bp, Penicillium crysogenum
  sig_peptide (1)..(54)
atgacactttcaaagctatctacgctcctagcctctgccactcttgttgcaggccatggctatgtctccagcatcgtagccgatggcaaaaggtgagtcaatctaagaccaaagcctctatgcaaacaacgaactaatcacatcatcatcagctacactggctacctcgttgacaaatatgcctacatggcagaccctcctgacagcgtcggctggactacaacagcaaccgacctcggtttcgaggacggcaccgaataccaagatcccaacataatctgccaccgagacggagccaacgcagctttgagtgccactgtcaaagctggctccagcattgacctccaatggacgacctggccagactctcaccacggccctgtgattacctatctcgcctcgtgcaacggtgactgcaccacagtcgacaagaccacgctggaattcttcaagattgaagaagaaggtcttatcgatggcgcaaaccctcctggcacatgggctaccgatgacctgattgctgctgggggtaagtggactgtcaagatcccttccactatcgctccgggtaactatgtcatgcgtcacgagattattgctcttcactctgctggccagaccaatggcgctcagaactacccgcagtgcttcaacctggaggtcactggtggtggaagtgacacgccagatggcacacctggtatggagttgtataccaacactgatcctgggatcttgttcgatatttattcttcgctgacttcgtatgatattccgggaccggctttgtatgcctga

SEQ ID NO: 21 - DNA, 796 bp, Penicillium expansum
  sig_peptide (1)..(54)
atgacattttcaaagctatctttgctcctagcctccgcctcttttgtggcaggtcatggctacgtctccagcatcgtggcaaatggcaaaaagtaagctaaaacaattccaagtgtctctataccaataatcaactaacaacactatcagttacactggctacatcgttgacaaatacgcctacatgtcagatccccccgacagcgtcggctgggcaacaacggcaaccgacctcggttttgaagacggcaccgaataccaagatccgaacataatctgccaccgcggcggaattaacgcagctttgagtgcccctgtcaccgctggctccagcattgatatccaatggacgacctggccagactcgcaccatggtcctctggtgacataccttgcctcgtgcaacggtgactgcagcacagtcgacaagaccacgctgaaattcttcaagattgaagaagacggtctcatcgatggtacaactcctcccggtacatgggccactgatgatcttattgctgctggggggcagtggactgtgagaattccttctaccatcgctgcgggtaactatgtcatgcgccatgaaattatcgctctgcattctgctggccagtccaatggcgcccagaactacccgcagtgtttcaatttgcaggtcactggtggtggaagtgatgtacctgatggcacgcttggtgagaagttgtataccaacactgacccggggatcttgttcaatctatattccacgttgacttcgtatattatcccaggaccggctttatatgcttga

SEQ ID NO: 22 - DNA, 1347 bp, Penicillium rubens
  sig_peptide (1)..(57)
atgtctgtctcaaagatcgcgggtctcatgcttagctcggccgccatggtcgctggccacggtttcgtctcaggtgctgttgttgatggcacctaccacggaggttacctcgtcaacaactacccctacaatgacaaccaccccgagactatcggatgggccgagaaggctaccgatctcggcttcgtcgatggcagcggctactctggccctgacattatttgccacaaggaggccactcctggtgccatctccgccgaggtcaaggccggtggtgatgtcgagctgcagtggaccgagtggcccgagagccaccacggccccgtcatcaactatctggctaactgcaacggcgactgctccaaggttgacaagaagaccctcaagtggttcaagatcgctgagtccggtctgatcgacggcagcaacgcccccggtaaatgggcatctgacgagctcattgccaacaacaacagcgcctccgtgaccatccccagctccatcgccgctggcaactacgttctccgtcacgagatcatcgccctgcactccgctggccaggagaacggtgcccagaactacccccagtgtctcaacctgaaggtcaccggcggcggcagcgacgtccccgagggtgtcgtcggaactgagctctacaagcccgacgacgccggcattttggtcagcatctacaaccagctgaaggactacaccatccccggtcccgctctgtacaagggcgcctcctccggctccggctccaagaccaccgacgctgcctccaccgcaactaccgccagtgcctcaaccgtgagtgcctctcccatccagagctcctcttcgcaccacctcacccgcacccgcactgcccgtcccacttacagcccctccggcacccctactccctcatccagtgccgtgtctgctgcctcaacctccacccctattgggtctggtgctgagtccaccgacgctccttcggtcgtcactgagactgccactgccactgcttcttctgctcctcaggttactgacgagcctgctacacagaccgccagcgaccccgccgacagcgtttccatcaccaccagcgccggtgcccagtctgcccagagctccgtccccgcctccggtggtgaggcctccgagcctagcgctacccctgcccccagctcctccggctctagctccggctccagctccagctccggatcttcctccgagtccggcgactacgcctcctacctcagctccctgagcgccgagaagctcctcgaagtcatccgctccaccctcaagtggcttgtctctgacaacaaggtgcacgcccgtgccctggctcactaa

SEQ ID NO: 23 - protein, 364 aa, Byssochlamys spectabilis
  SIGNAL (1)..(21)
MPFSKIAILATILGTASLVSGHGYVQGIVADGKYYGGYIVTQYPYTNDPPELVAWSTEATDLGFVDGSGYASGDIICHKSAQPAALAAEVPAGGKVELQWTEWPESHHGPVLDYLAPCNGNCADVDKTALEFFKIDQGGLVDDSSPPGTWASDQLISNNNSWTVTIPSTIAPGNYVLRHEIIALHSAGNKDGAQNYPQCINIKVTGSGTDSPSGTLGTSLYKDTDPGILVNIYQTLSDYSIPGPALYEGGSSSGTSSGSGTSTATSTSVASSTSVKPSSISTPSSVQVAIPSAKTSAVPATTQAPTTATPAVTIPTESALTDYLNSMSADEVLNLVRGTMSWLFTNKNHARDLSASSELTDRPM

SEQ ID NO: 24 - protein, 249 aa, Penicillium oxalicum
  SIGNAL (1)..(21)
MVSSKATTLSAMLACVSMVAGHGFVSSIMAGGKNYTGYLANSYPYMSNPPQSVGWATTATDLGFEDGTEYQSANIICHRDGKNAALSAPVTAGSKVEIQWTSWPDSHHGPVITYLASCGGDCSTVDKTTLKFFKIDEAGLIDDSNPPGTWATDQLIAAGNRWTVTIPKSIASGNYVMRHEIIALHSSGSKDGAQNYPQCLNLQVTGGGSAKPQGTLGEALYKDTDPGILINIYQSLKSYVIPGPALYSA

SEQ ID NO: 25 - protein, 343 aa, Aspergillus clavatus
  SIGNAL (1)..(19)
MSVTKIAGILLGSAAMVAGHGFVTGAVVDGTYHTGYLVNNYPYSSNPPKTIGWSETATDLGFVDGTGYASGDIICHKNAKPGALSADIKAGGKVEFQWTQWPESHHGPVITYMANCNGDCASVDKSQLEFFKIEEKGLIGSGHWASDDLIANNNSWTVTVPSSLAAGNYVMRHEIIGLHSAGNANGAQNYPQCINLKVTGGGSDKPVGTLGTKLYKATDPGILVNIYGSLTSYTIPGPALWTGASSGGSDDNGSTPASTPTPTAAPSSTAAPSVTITSTPGQKTQTPVATPAPSTPSSTPGLPTDGNLTDYFKSLSPEQFLSVLKETFSWLVTEKVHARSLSA

SEQ ID NO: 26 - protein, 373 aa, Aspergillus fumigatus
  SIGNAL (1)..(19)
MSVPKIAAALLSSAALVAGHGFVTGAVVDGKYYTGYLVNQYPYMSSPPDSIGWSETATDLGFVDGSGYSSGDIICHKDAKNGAISAEIKAGGKVEFQWTEWPESHHGPVITYMANCNGDCASVDKTTLEFFKIDESGLISDSNVPGTWASDNLIANNNSWTVTVPSSIAAGNYVMRHEIIALHSAGNQNGAQNYPQCINLKVTGGGSDKPAGTLGTALYKNTDAGILVNIYQSLSSYEIPGPALYSGASSGSSNNGGSASSSATAPSATITQPSTAVPTSSATAYQPSTTTEAVTVTSIPAQQSYVQAPTATPSSTAGSSGSGSGSSSSGTLPSSSNLTEYFNSLSAEEFLKVLKQTFSWLVTEKVHARDLSA

SEQ ID NO: 27 - protein, 402 aa, Aspergillus niger
  SIGNAL (1)..(19)
MSVAKIAGVVLGSAALVAGHGYVSGAVIDGEYYGGYIVSSYPYESDPPETIAWSTTATDLGFVDGSEYSDPDIICHKSAKPGAISADVKAGGTVELQWTDWPSSHHGPVLTYLANCNGDCSDVTKTDLEFFKIDESGLISDTEVPGTWATDNLISNNNSWTVTIPSTLEAGNYVLRHEIIALHSAENKDGAQNYPQCLNLKVTGSGSSTYSGTKGEALYKDTDPGILVNIYETLSSYDIPGPAMYNATSSSSSSGSSSGASSTSSAAAAAATTSSASTSSASTPTTSAAAATSSAVSATTTAAAAVVTDVETTVQTAVATAYQTSTDVVQVTVTGSAPQQTHIQVASSSAAASTPSSSSVAVSSDNLTSYFSSLSADQFLSVLKETFSWLVTEKKHARDLNA

SEQ ID NO: 28 - protein, 353 aa, Aspergillus niger
  SIGNAL (1)..(19)
MSVAKIAGVVLGSAALVAGHGYVSGAVIDGEYYGGYIVSSYAYESDPPETIAWSTTATDLGFVDGSEYSDPDIICHKSAKPGAISADVKAGGTVELQWTDWPSSHHGPVLTYLANCNGDCSDVTKTDLEFFKIDESGLISDTEVPGTWATDNLISNNNSWTVTIPSTLEAGNYVLRHEIIALHSAENKDGAQNYPQCLNLKVTGSGSSTYSGTKGEALYKDTDPGILVNIYETLSSYDIPGPAISAVSATTTAAAAVVTDVETTVQTAVATAYQTSTDVVQVTVTGSAPQQTHIQVASSSAAASTPSSSSVAVSSDNLTSYFSSLSADQFLSVLKETFSWLVTEKKHARDLNA

SEQ ID NO: 29 - protein, 344 aa, Aspergillus ruber
  SIGNAL (1)..(19)
MSVTKITGLLLGSAAMVAGHGYVSGAVVDGQYYGGYIVTSYAYTDSPPETIGWSTEATDLGFVSPSSFSDPDIICHKSAQPGAISASVAAGKDVELQWTEWPESHHGPVITYLANCNGDCSKVDKTSLEFFKIDQKGLVDDSNVPGTWASDNLISNNNSYTVTIPSDIAAGNYVLRHEIIALHSAGNEDGAQSYPQCVNLKVTGGGSASPSGTLGTKLYSEDDPGILVNIYQQLDSYEIPGPALYSGASSSSNSGSSSSTASASTTATSATTSSASSTQASATPASQAKAQVSSSTPSASSVATSGSLSDYFSSLSAEEFLNVISETLSWLVTDKIHARDISTA

SEQ ID NO: 30 - protein, 402 aa, Aspergillus terreus
  SIGNAL (1)..(19)
MSVAKIAGVVLGSAALVAGHGYVTGAVVDGKYYAGYDVTSAPYSSDPPATIGWSTTATDLGFVDGTEYSEPDIICHKDAKPGSLSAEITAGGKVELQWTEWPESHHGPVITYLANCNGDCSSVDKTSLEFFKIDQKGLIEGNTWASDQLISNNNSWTVTIPSSIASGNYVLRHEIIGLHSAGNKDGAQNYPQCMNLKVTGGGSDKPAGTLGTALYKDTDPGILVNIYQTLSSYVIPGPALYSGSSSGSGSGSASSSAAATPSSSSVPSSTSIAVPTSSAPAPTSAAPSAQTSTATAYQTATTIESVTVTAEAPQQTKVQDPSSGSSSGSNSGSSSGSSSGSSTGSSSGSSTGTGSSSVPTPSGSSSLSDYFNSLSAEQFLNLLRQTLSWLITDKKHARDLSA

SEQ ID NO: 31 - protein, 367 aa, Neosartorya fischeri
  SIGNAL (1)..(19)
MSVSKIAAVLLSSAALVAGHGFVSGAVVDGKYYTGYLVNQYPYMSSPPESIGWSETATDLGFVDGSGYSSGDIICHKSAKNGAISAEIKAGGKVEFQWTEWPESHHGPVITYMANCNGDCASVDKTSLEFFKIDESGLISDSNVPGTWASDNLIANNNSWTVTVPSSIAAGNYVMRHEIIALHSAGNQNGAQNYPQCINLKVTGGGSDKPAGTLGTALYKNTDAGILVNIYQSLSSYDIPGPALYSGASSGSSNSGGSASSSAAAPSATITQTSTAVQTSSATVYQPSTTTEAVTVTSTPAQQSYVQAPTATPSSTAGSSSSSGTLPSGSSLTEYFNSLSAEEFLKVLKETFSWLVTEKVHARDLSA

SEQ ID NO: 32 - protein, 400 aa, Aspergillus kawachii
  SIGNAL (1)..(19)
MSVAKIAGVVLGSAALVAGHGYVSGAVVDGQYYGGYIVSSYAYESDPPETIAWSTEATDLGFVDGSEYAEPDIICHKSAKPGAISADVKAGGTVELQWTDWPSSHHGPVLTYLANCNGDCSDVTKTDLEFFKIDESGLISDTEVPGTWATDNLISNNNSWTVTIPSTLESGNYVLRHEIIALHSAGNKDGAQNYPQCLNLKVTGSGSSTYSGTKGEALYKDTDPGILVNIYQSLSSYDIPGPAMYNATSSGSSSGSSSSASSTSSAAAAAATTSSASSSTTSAAAATSSAVSATTTSAAAAAVVTDVETTVQTAVATAYQTSTDVVQVTVTGSAPQQTHIQVASSSAAAVATPSSSSVAVSSDNLTSYFSSLSADQFLSVLKETFSWLVTEKKHARDLNA

SEQ ID NO: 33 - protein, 434 aa, Aspergillus nidulans
  SIGNAL (1)..(19)
MSVARTAGFALASAAIVAGHGYVTGIVADGTYYGGYLVNQYPYSNDPPAVVGWAEDATDLGFVDGSGYTSGDIICHKDATNAQASATVAAGGTVELQWTEWPESHHGPVIDYIASCNGDCTTVDKTTLEWVKISESGLVDGSSAPGTWASDNLISNNNSWTVTIPSSLAAGGYVLRHEIIALHSAGNENGAQNYPQCVNLEVTGGGSASPSGTVGTELYTPTDPGILVNIYTSLDSYTIPGPALWDGASSSGGNSGSGSASSSAAATSTPTTPSVSVPVIPTASSGASSTPLVPTPSAPAVTPSVPAGNQAPQPTYTSTYIETETLPGQTVTSTTTEYASEPTQPAVETQVAQPSETEAATSTSTVTETASATAAPTGSSGSSSGSGSSSTELPTDSSSLSDYFSSLSAEEFLNLLKETLKWLVTDKVHARSLH

SEQ ID NO: 34 - protein, 250 aa, Aspergillus oryzae
  SIGNAL (1)..(21)
MAMSKIVSLTGLLASASLVAGHGYVSGVVIDGQYYGGYLVDKYAYSDNAPDTIGWTTSATDLGFVDGTGYQSPDIICHKDGAPGALTAEVAAGGKIELQWTEWPESHHGPVLNYLAPCGGECSAVDKTTLEFFKIEAKGLIDGTTPPGQWATDDLISNNNSYTVTIPTSIQEGNYVLRHEIIGLHSAGQKDGAQNYPQCINIKVTGGGDATPAGTAGEALYKDTDAGILFDIYSDLSGGYPIPGPEVFSA

SEQ ID NO: 35 - protein, 370 aa, Aspergillus oryzae
  SIGNAL (1)..(19)
MSIAKIAGVVLGSAALVAGHGYVSGAVVDGQYYSGYDMSYHYMSNPPQVIGWSTDATDLGFVDGSSYADADIICHKNAKNGAISAEIAAGKQVELQWTAWPESHKGPVITYLANCNGDCATVDKTQLEFFKIDEKGLISGSDNTWASDNLISSNNSWTVTIPSSIAAGNYVMRHEIIALHSAGNKDGAQNYPQCLNFKVTGGGSDKPEGTLGTALYKDTDPGIEINIYQTLSSYTIPGPALYTGSSSSGSSGSGSSSAAPSPTASASASATAAPVQTSTATAYQTSTAVASVTVTGSAPAQTHVQATSSSAAASTPTASSGASTGSGSGSSSSDLTSYFNSLSADELLNVIKQTLSWLVTDKIHARDISA

SEQ ID NO: 36 - protein, 378 aa, Baudoinia compniacensis
  SIGNAL (1)..(19)
MSVIQSAAILASLVASVAAHGHVQGIVAGGTYYTGYDPSFQYSPIPPIVIGWSDPQDLDNGFIAPSNYTNPDIICHRNATAAGTSARIAAGDVVEPQWTPWPSSHHGPVIDYLAKCPGSCSDVDKTTLEFFKIDQVGLIDDTTEPGTWGSDQLLANNNSWTVTIPPSIAPGNYVLRHEIIALHSAGTADGAQNYPQCVNLEITGSGTDTPAGTLGQKLYGANDSGILINIYQSLSTYEIPGPSLYSGAVAMSQTQVAITGYAPLSSGGVAVSIAYSSATTTTAAAGSSSVARSTTAASASASTAIVSALPSTALTQTLPTVLPTGTAVSSNDTIAGLPPAEAPPAGTTLQDLFAWLEYMLEGLASTGGRHQRDFKERK

SEQ ID NO: 37 - protein, 245 aa, Penicillium roqueforti
  SIGNAL (1)..(18)
MTLSKLSTLLASASLVAGHGYVSSIVADGKNYTGYIVDKYPYMADPPDSVGWATTATDLGFEDGTEYQDPNIICHRNGVNAALSATVTAGSSIDLQWTTWPDSHHGPLITYLASCNGDCSTVDKTTLKFFKIEEDGLIDGATPPGTWATDDLIAAGGQWTVKIPPTLAAGNYVMRHEIIALHSAGQLNGAQNYPQCFNLEITGGGSDVPDGTFGGELYINTDPGILFDIYSTLTSYIIPGPALYA

SEQ ID NO: 38 - protein, 318 aa, Sclerotinia sclerotiorum
  SIGNAL (1)..(18)
MSFSKLTTFAGLVATVAAHGYVTGIVAGGTYTKGWLVNYAYQNPMPESIGWSDQNLDLGFTAPSAYATGDIICAKNGTNAALSAKVAAGDTVDFQWTVWPDSHKGPVITYLANCNGDCSTVDKTKLEFFKIDEGGLIDDTTVPGTWASDNLITNNNTWTSTIPADLAPGNYVARHEIIALHGAGSNDGAQNYPQCINLEVTGSGTLAPSGTLGEKLYTPTDPGILVNIYAKLTYTIPGPPMYNATGAATGAATGATTPATGKTASATSAAAALSTPAVVSTPPTPTTMITSVAPAATPATSAAASSGASGDDDDTCEA

SEQ ID NO: 39 - protein, 404 aa, Penicillium roqueforti
  SIGNAL (1)..(19)
MSVSKIAGLLLSSAAMVAGHGFVSGAVVDGTYHGGFIVNTYNYMTEVPANIGWSEKATDLGYIDGSGYTGSDIICHKEATPGAISAEVKAGGSVELQWTDWPESHHGPVITYMANCNGDCSVVDKTTLKFFKIAEAGLIDDTNVPGTWASDQLIAANNSATVTIPSTIAAGNYVLRHEIIALHSAGDANGAQNYPQCINLKVTGGGSDSPDGVLGTALYTPTDPGILISIYSALESYVIPGPALYTGASSGSSSTSSTTAAATTEATTASATVSTTAAPTVATTSASTTASTADSITITTTAGGLSAQSSAAVLESSATSTVTAEATATAAPTAAATTSTSSTSSGSSSSSSTSSSGSSSTSSTSTSNSSYLSSLSAEKLLEVIRSTLKWLVSDKKVHARALAY

SEQ ID NO: 40 - protein, 245 aa, Penicillium italicum
  SIGNAL (1)..(18)
MTLSKLSMLLASASLAAGHGYVSSIVANGKNYTGYIVDKYAYMSDPPDSVGWATTATDLGFEDGTEYQNPNIICHRGGVNAALSATVAAGSSIDIQWTTWPDSHHGPLITYLASCNGDCSTVDKTTLKFFKIEEDGLIHGATPPGTWATDDLIASGGQWTVRIPSTIATGNYVMRHEIIALHSAGQLNGAQNYPQCFNLQVTGGGGNAPNGTLGEKLYTNTDPGILFNIYSTLTSYVIPGPALYA

SEQ ID NO: 41 - protein, 372 aa, Aspergillus flavus
  SIGNAL (1)..(20)
MSIAKIAGIVLGSAALVAAHGHVSGAVVDGQYYPGYDISYHYMPDPPKVIGWSTDATDNGFVDGSSYADADIICHKNAKNGAISAEIAAGKQVELQWTAWPESHKGPVITYLANCNGDCATVDKTQLEFFKIDEKGLISGSDNTWATDNLIASNNSWTVTIPSSIAAGNYVMRHEIIGLHSAGQKDGAQNYPQCLNFKVTGGGSDKPEGTLGTALYKDTDPGIQINIYQTLTSYTIPGPALYSGSSSSGSSGSGSSSAAPSATASASASASATAAPVQTSTATAYQTSTAVASVTVTGSAPAQTHVQATSSSAAASTPTASSGASTGSGSGSSSSDLTGYFNSLSADELLNVIKQTLSWLVTDKIHARDISA

SEQ ID NO: 42 - protein, 245 aa, Penicillium crysogenum
  SIGNAL (1)..(18)
MTLSKLSTLLASATLVAGHGYVSSIVADGKSYTGYLVDKYAYMADPPDSVGWTTTATDLGFEDGTEYQDPNIICHRDGANAALSATVKAGSSIDLQWTTWPDSHHGPVITYLASCNGDCTTVDKTTLEFFKIEEEGLIDGANPPGTWATDDLIAAGGKWTVKIPSTIAPGNYVMRHEIIALHSAGQTNGAQNYPQCFNLEVTGGGSDTPDGTPGMELYTNTDPGILFDIYSSLTSYDIPGPALYA

SEQ ID NO: 43 - protein, 245 aa, Penicillium expansum
  SIGNAL (1)..(18)
MTFSKLSLLLASASFVAGHGYVSSIVANGKNYTGYIVDKYAYMSDPPDSVGWATTATDLGFEDGTEYQDPNIICHRGGINAALSAPVTAGSSIDIQWTTWPDSHHGPLVTYLASCNGDCSTVDKTTLKFFKIEEDGLIDGTTPPGTWATDDLIAAGGQWTVRIPSTIAAGNYVMRHEIIALHSAGQSNGAQNYPQCFNLQVTGGGSDVPDGTLGEKLYTNTDPGILFNLYSTLTSYIIPGPALYA

SEQ ID NO: 44 - protein, 393 aa, Penicillium rubens
  SIGNAL (1)..(19)
MSVSKIAGLMLSSAAMVAGHGFVSGAVVDGTYHGGYLVNNYPYNDNHPETIGWAEKATDLGFVDGSGYSGPDIICHKEATPGAISAEVKAGGDVELQWTEWPESHHGPVINYLANCNGDCSKVDKKTLKWFKIAESGLIDGSNAPGKWASDELIANNNSASVTIPSSIAAGNYVLRHEIIALHSAGQENGAQNYPQCLNLKVTGGGSDVPEGVVGTELYKPDDAGILVSIYNQLKDYTIPGPALYKGASSGSGSKTTDAASTATTASASTVSASPIQSSSSHHLTRTRTARPTYSPSGTPTPSSSAVSAASTSTPIGSVPASGGEASEPSATPAPSSSGSSSGSSSSSGSSSESGDYASYLSSLSAEKLLEVIRSTLKWLVSDNKVHARALAH

SEQ ID NO: 45 - DNA, 54 bp, Aspergillus niger
atgtcgttccgatctcttctcgccctgagcggccttgtctgctcggggttggca

SEQ ID NO: 46 - protein, 18 aa, Aspergillus niger
MSFRSLLALSGLVCSGLA

SEQ ID NO: 47 - DNA, 54 bp, Myceliophthora thermophila
atgaagtccttcaccctcaccactctggccgccctggctggcaacgccgccgct

SEQ ID NO: 48 - protein, 18 aa, Myceliophthora thermophila
MKSFTLTTLAALAGNAAA

SEQ ID NO: 49 - protein, 343 aa, Byssochlamys spectabilis
HGYVQGIVADGKYYGGYIVTQYPYTNDPPELVAWSTEATDLGFVDGSGYASGDIICHKSAQPAALAAEVPAGGKVELQWTEWPESHHGPVLDYLAPCNGNCADVDKTALEFFKIDQGGLVDDSSPPGTWASDQLISNNNSWTVTIPSTIAPGNYVLRHEIIALHSAGNKDGAQNYPQCINIKVTGSGTDSPSGTLGTSLYKDTDPGILVNIYQTLSDYSIPGPALYEGGSSSGTSSGSGTSTATSTSVASSTSVKPSSISTPSSVQVAIPSAKTSAVPATTQAPTTATPAVTIPTESALTDYLNSMSADEVLNLVRGTMSWLFTNKNHARDLSASSELTDRPM

SEQ ID NO: 50 - protein, 228 aa, Penicillium oxalicum
HGFVSSIMAGGKNYTGYLANSYPYMSNPPQSVGWATTATDLGFEDGTEYQSANIICHRDGKNAALSAPVTAGSKVEIQWTSWPDSHHGPVITYLASCGGDCSTVDKTTLKFFKIDEAGLIDDSNPPGTWATDQLIAAGNRWTVTIPKSIASGNYVMRHEIIALHSSGSKDGAQNYPQCLNLQVTGGGSAKPQGTLGEALYKDTDPGILINIYQSLKSYVIPGPALYSA

SEQ ID NO: 51 - protein, 324 aa, Aspergillus clavatus
HGFVTGAVVDGTYHTGYLVNNYPYSSNPPKTIGWSETATDLGFVDGTGYASGDIICHKNAKPGALSADIKAGGKVEFQWTQWPESHHGPVITYMANCNGDCASVDKSQLEFFKIEEKGLIGSGHWASDDLIANNNSWTVTVPSSLAAGNYVMRHEIIGLHSAGNANGAQNYPQCINLKVTGGGSDKPVGTLGTKLYKATDPGILVNIYGSLTSYTIPGPALWTGASSGGSDDNGSTPASTPTPTAAPSSTAAPSVTITSTPGQKTQTPVATPAPSTPSSTPGLPTDGNLTDYFKSLSPEQFLSVLKETFSWLVTEKVHARSLSA

SEQ ID NO: 52 - protein, 354 aa, Aspergillus fumigatus
HGFVTGAVVDGKYYTGYLVNQYPYMSSPPDSIGWSETATDLGFVDGSGYSSGDIICHKDAKNGAISAEIKAGGKVEFQWTEWPESHHGPVITYMANCNGDCASVDKTTLEFFKIDESGLISDSNVPGTWASDNLIANNNSWTVTVPSSIAAGNYVMRHEIIALHSAGNQNGAQNYPQCINLKVTGGGSDKPAGTLGTALYKNTDAGILVNIYQSLSSYEIPGPALYSGASSGSSNNGGSASSSATAPSATITQPSTAVPTSSATAYQPSTTTEAVTVTSIPAQQSYVQAPTATPSSTAGSSGSGSGSSSSGTLPSSSNLTEYFNSLSAEEFLKVLKQTFSWLVTEKVHARDLSA

SEQ ID NO: 53 - protein, 383 aa, Aspergillus niger
HGYVSGAVIDGEYYGGYIVSSYPYESDPPETIAWSTTATDLGFVDGSEYSDPDIICHKSAKPGAISADVKAGGTVELQWTDWPSSHHGPVLTYLANCNGDCSDVTKTDLEFFKIDESGLISDTEVPGTWATDNLISNNNSWTVTIPSTLEAGNYVLRHEIIALHSAENKDGAQNYPQCLNLKVTGSGSSTYSGTKGEALYKDTDPGILVNIYETLSSYDIPGPAMYNATSSSSSSGSSSGASSTSSAAAAAATTSSASTSSASTPTTSAAAATSSAVSATTTAAAAVVTDVETTVQTAVATAYQTSTDVVQVTVTGSAPQQTHIQVASSSAAASTPSSSSVAVSSDNLTSYFSSLSADQFLSVLKETFSWLVTEKKHARDLNA

SEQ ID NO: 54 - protein, 334 aa, Aspergillus niger
HGYVSGAVIDGEYYGGYIVSSYAYESDPPETIAWSTTATDLGFVDGSEYSDPDIICHKSAKPGAISADVKAGGTVELQWTDWPSSHHGPVLTYLANCNGDCSDVTKTDLEFFKIDESGLISDTEVPGTWATDNLISNNNSWTVTIPSTLEAGNYVLRHEIIALHSAENKDGAQNYPQCLNLKVTGSGSSTYSGTKGEALYKDTDPGILVNIYETLSSYDIPGPAISAVSATTTAAAAVVTDVETTVQTAVATAYQTSTDVVQVTVTGSAPQQTHIQVASSSAAASTPSSSSVAVSSDNLTSYFSSLSADQFLSVLKETFSWLVTEKKHARDLNA

SEQ ID NO: 55 - protein, 325 aa, Aspergillus ruber
HGYVSGAVVDGQYYGGYIVTSYAYTDSPPETIGWSTEATDLGFVSPSSFSDPDIICHKSAQPGAISASVAAGKDVELQWTEWPESHHGPVITYLANCNGDCSKVDKTSLEFFKIDQKGLVDDSNVPGTWASDNLISNNNSYTVTIPSDIAAGNYVLRHEIIALHSAGNEDGAQSYPQCVNLKVTGGGSASPSGTLGTKLYSEDDPGILVNIYQQLDSYEIPGPALYSGASSSSNSGSSSSTASASTTATSATTSSASSTQASATPASQAKAQVSSSTPSASSVATSGSLSDYFSSLSAEEFLNVISETLSWLVTDKIHARDISTA

SEQ ID NO: 56 - protein, 383 aa, Aspergillus terreus
HGYVTGAVVDGKYYAGYDVTSAPYSSDPPATIGWSTTATDLGFVDGTEYSEPDIICHKDAKPGSLSAEITAGGKVELQWTEWPESHHGPVITYLANCNGDCSSVDKTSLEFFKIDQKGLIEGNTWASDQLISNNNSWTVTIPSSIASGNYVLRHEIIGLHSAGNKDGAQNYPQCMNLKVTGGGSDKPAGTLGTALYKDTDPGILVNIYQTLSSYVIPGPALYSGSSSGSGSGSASSSAAATPSSSSVPSSTSIAVPTSSAPAPTSAAPSAQTSTATAYQTATTIESVTVTAEAPQQTKVQDPSSGSSSGSNSGSSSGSSSGSSTGSSSGSSTGTGSSSVPTPSGSSSLSDYFNSLSAEQFLNLLRQTLSWLITDKKHARDLSA

SEQ ID NO: 57 - protein, 348 aa, Neosartorya fischeri
HGFVSGAVVDGKYYTGYLVNQYPYMSSPPESIGWSETATDLGFVDGSGYSSGDIICHKSAKNGAISAEIKAGGKVEFQWTEWPESHHGPVITYMANCNGDCASVDKTSLEFFKIDESGLISDSNVPGTWASDNLIANNNSWTVTVPSSIAAGNYVMRHEIIALHSAGNQNGAQNYPQCINLKVTGGGSDKPAGTLGTALYKNTDAGILVNIYQSLSSYDIPGPALYSGASSGSSNSGGSASSSAAAPSATITQTSTAVQTSSATVYQPSTTTEAVTVTSTPAQQSYVQAPTATPSSTAGSSSSSGTLPSGSSLTEYFNSLSAEEFLKVLKETFSWLVTEKVHARDLSA

SEQ ID NO: 58 - protein, 381 aa, Aspergillus kawachii
HGYVSGAVVDGQYYGGYIVSSYAYESDPPETIAWSTEATDLGFVDGSEYAEPDIICHKSAKPGAISADVKAGGTVELQWTDWPSSHHGPVLTYLANCNGDCSDVTKTDLEFFKIDESGLISDTEVPGTWATDNLISNNNSWTVTIPSTLESGNYVLRHEIIALHSAGNKDGAQNYPQCLNLKVTGSGSSTYSGTKGEALYKDTDPGILVNIYQSLSSYDIPGPAMYNATSSGSSSGSSSSASSTSSAAAAAATTSSASSSTTSAAAATSSAVSATTTSAAAAAVVTDVETTVQTAVATAYQTSTDVVQVTVTGSAPQQTHIQVASSSAAAVATPSSSSVAVSSDNLTSYFSSLSADQFLSVLKETFSWLVTEKKHARDLNA

SEQ ID NO: 59 - protein, 415 aa, Aspergillus nidulans
HGYVTGIVADGTYYGGYLVNQYPYSNDPPAVVGWAEDATDLGFVDGSGYTSGDIICHKDATNAQASATVAAGGTVELQWTEWPESHHGPVIDYIASCNGDCTTVDKTTLEWVKISESGLVDGSSAPGTWASDNLISNNNSWTVTIPSSLAAGGYVLRHEIIALHSAGNENGAQNYPQCVNLEVTGGGSASPSGTVGTELYTPTDPGILVNIYTSLDSYTIPGPALWDGASSSGGNSGSGSASSSAAATSTPTTPSVSVPVIPTASSGASSTPLVPTPSAPAVTPSVPAGNQAPQPTYTSTYIETETLPGQTVTSTTTEYASEPTQPAVETQVAQPSETEAATSTSTVTETASATAAPTGSSGSSSGSGSSSTELPTDSSSLSDYFSSLSAEEFLNLLKETLKWLVTDKVHARSLH

SEQ ID NO: 60 - protein, 229 aa, Aspergillus oryzae
HGYVSGVVIDGQYYGGYLVDKYAYSDNAPDTIGWTTSATDLGFVDGTGYQSPDIICHKDGAPGALTAEVAAGGKIELQWTEWPESHHGPVLNYLAPCGGECSAVDKTTLEFFKIEAKGLIDGTTPPGQWATDDLISNNNSYTVTIPTSIQEGNYVLRHEIIGLHSAGQKDGAQNYPQCINIKVTGGGDATPAGTAGEALYKDTDAGILFDIYSDLSGGYPIPGPEVFSA

SEQ ID NO: 61 - protein, 351 aa, Aspergillus oryzae
HGYVSGAVVDGQYYSGYDMSYHYMSNPPQVIGWSTDATDLGFVDGSSYADADIICHKNAKNGAISAEIAAGKQVELQWTAWPESHKGPVITYLANCNGDCATVDKTQLEFFKIDEKGLISGSDNTWASDNLISSNNSWTVTIPSSIAAGNYVMRHEIIALHSAGNKDGAQNYPQCLNFKVTGGGSDKPEGTLGTALYKDTDPGIEINIYQTLSSYTIPGPALYTGSSSSGSSGSGSSSAAPSPTASASASATAAPVQTSTATAYQTSTAVASVTVTGSAPAQTHVQATSSSAAASTPTASSGASTGSGSGSSSSDLTSYFNSLSADELLNVIKQTLSWLVTDKIHARDISA

SEQ ID NO: 62 - protein, 359 aa, Baudoinia compniacensis
HGHVQGIVAGGTYYTGYDPSFQYSPIPPIVIGWSDPQDLDNGFIAPSNYTNPDIICHRNATAAGTSARIAAGDVVEPQWTPWPSSHHGPVIDYLAKCPGSCSDVDKTTLEFFKIDQVGLIDDTTEPGTWGSDQLLANNNSWTVTIPPSIAPGNYVLRHEIIALHSAGTADGAQNYPQCVNLEITGSGTDTPAGTLGQKLYGANDSGILINIYQSLSTYEIPGPSLYSGAVAMSQTQVAITGYAPLSSGGVAVSIAYSSATTTTAAAGSSSVARSTTAASASASTAIVSALPSTALTQTLPTVLPTGTAVSSNDTIAGLPPAEAPPAGTTLQDLFAWLEYMLEGLASTGGRHQRDFKERK

SEQ ID NO: 63 - protein, 227 aa, Penicillium roqueforti
HGYVSSIVADGKNYTGYIVDKYPYMADPPDSVGWATTATDLGFEDGTEYQDPNIICHRNGVNAALSATVTAGSSIDLQWTTWPDSHHGPLITYLASCNGDCSTVDKTTLKFFKIEEDGLIDGATPPGTWATDDLIAAGGQWTVKIPPTLAAGNYVMRHEIIALHSAGQLNGAQNYPQCFNLEITGGGSDVPDGTFGGELYINTDPGILFDIYSTLTSYIIPGPALYA

SEQ ID NO: 64 - protein, 300 aa, Sclerotinia sclerotiorum
HGYVTGIVAGGTYTKGWLVNYAYQNPMPESIGWSDQNLDLGFTAPSAYATGDIICAKNGTNAALSAKVAAGDTVDFQWTVWPDSHKGPVITYLANCNGDCSTVDKTKLEFFKIDEGGLIDDTTVPGTWASDNLITNNNTWTSTIPADLAPGNYVARHEIIALHGAGSNDGAQNYPQCINLEVTGSGTLAPSGTLGEKLYTPTDPGILVNIYAKLTYTIPGPPMYNATGAATGAATGATTPATGKTASATSAAAALSTPAVVSTPPTPTTMITSVAPAATPATSAAASSGASGDDDDTCEA

SEQ ID NO: 65 - protein, 385 aa, Penicillium roqueforti
HGFVSGAVVDGTYHGGFIVNTYNYMTEVPANIGWSEKATDLGYIDGSGYTGSDIICHKEATPGAISAEVKAGGSVELQWTDWPESHHGPVITYMANCNGDCSVVDKTTLKFFKIAEAGLIDDTNVPGTWASDQLIAANNSATVTIPSTIAAGNYVLRHEIIALHSAGDANGAQNYPQCINLKVTGGGSDSPDGVLGTALYTPTDPGILISIYSALESYVIPGPALYTGASSGSSSTSSTTAAATTEATTASATVSTTAAPTVATTSASTTASTADSITITTTAGGLSAQSSAAVLESSATSTVTAEATATAAPTAAATTSTSSTSSGSSSSSSTSSSGSSSTSSTSTSNSSYLSSLSAEKLLEVIRSTLKWLVSDKKVHARALAY

SEQ ID NO: 66 - protein, 227 aa, Penicillium italicum
HGYVSSIVANGKNYTGYIVDKYAYMSDPPDSVGWATTATDLGFEDGTEYQNPNIICHRGGVNAALSATVAAGSSIDIQWTTWPDSHHGPLITYLASCNGDCSTVDKTTLKFFKIEEDGLIHGATPPGTWATDDLIASGGQWTVRIPSTIATGNYVMRHEIIALHSAGQLNGAQNYPQCFNLQVTGGGGNAPNGTLGEKLYTNTDPGILFNIYSTLTSYVIPGPALYA

SEQ ID NO: 67 - protein, 352 aa, Aspergillus flavus
GHVSGAVVDGQYYPGYDISYHYMPDPPKVIGWSTDATDNGFVDGSSYADADIICHKNAKNGAISAEIAAGKQVELQWTAWPESHKGPVITYLANCNGDCATVDKTQLEFFKIDEKGLISGSDNTWATDNLIASNNSWTVTIPSSIAAGNYVMRHEIIGLHSAGQKDGAQNYPQCLNFKVTGGGSDKPEGTLGTALYKDTDPGIQINIYQTLTSYTIPGPALYSGSSSSGSSGSGSSSAAPSATASASASASATAAPVQTSTATAYQTSTAVASVTVTGSAPAQTHVQATSSSAAASTPTASSGASTGSGSGSSSSDLTGYFNSLSADELLNVIKQTLSWLVTDKIHARDISA

SEQ ID NO: 68 - protein, 227 aa, Penicillium crysogenum
HGYVSSIVADGKSYTGYLVDKYAYMADPPDSVGWTTTATDLGFEDGTEYQDPNIICHRDGANAALSATVKAGSSIDLQWTTWPDSHHGPVITYLASCNGDCTTVDKTTLEFFKIEEEGLIDGANPPGTWATDDLIAAGGKWTVKIPSTIAPGNYVMRHEIIALHSAGQTNGAQNYPQCFNLEVTGGGSDTPDGTPGMELYTNTDPGILFDIYSSLTSYDIPGPALYA

SEQ ID NO: 69 - protein, 227 aa, Penicillium expansum
HGYVSSIVANGKNYTGYIVDKYAYMSDPPDSVGWATTATDLGFEDGTEYQDPNIICHRGGINAALSAPVTAGSSIDIQWTTWPDSHHGPLVTYLASCNGDCSTVDKTTLKFFKIEEDGLIDGTTPPGTWATDDLIAAGGQWTVRIPSTIAAGNYVMRHEIIALHSAGQSNGAQNYPQCFNLQVTGGGSDVPDGTLGEKLYTNTDPGILFNLYSTLTSYIIPGPALYA

SEQ ID NO: 70 - protein, 374 aa, Penicillium rubens
HGFVSGAVVDGTYHGGYLVNNYPYNDNHPETIGWAEKATDLGFVDGSGYSGPDIICHKEATPGAISAEVKAGGDVELQWTEWPESHHGPVINYLANCNGDCSKVDKKTLKWFKIAESGLIDGSNAPGKWASDELIANNNSASVTIPSSIAAGNYVLRHEIIALHSAGQENGAQNYPQCLNLKVTGGGSDVPEGVVGTELYKPDDAGILVSIYNQLKDYTIPGPALYKGASSGSGSKTTDAASTATTASASTVSASPIQSSSSHHLTRTRTARPTYSPSGTPTPSSSAVSAASTSTPIGSVPASGGEASEPSATPAPSSSGSSSGSSSSSGSSSESGDYASYLSSLSAEKLLEVIRSTLKWLVSDNKVHARALAH